(19) United States
(12) Patent Application Publication
(10) Pub. No.: US 2024/0293524 A1
(43) Pub. Date: Sep. 5, 2024

(54) MULTIPLE FUNCTIONALIZED noMV CONJUGATES

(71) Applicant: GLAXOSMITHKLINE BIOLOGICALS SA, Rixensart (BE)

(72) Inventors: Renzo ALFINI, Siena (IT); Roberta Di Benedetto, Siena (IT); Francesca Micoli, Siena (IT); Allan James Saul, Siena (IT)

(73) Assignee: GLAXOSMITHKLINE BIOLOGICALS SA, Rixensart (BE)

(21) Appl. No.: 18/620,109

(22) Filed: Mar. 28, 2024

Related U.S. Application Data
(63) Continuation of application No. 16/636,560, filed on Feb. 4, 2020, now Pat. No. 12,005,109.

(30) Foreign Application Priority Data
Aug. 10, 2017 (GB) 1712824.0

(51) Int. Cl. A61K 39/095; A61K 35/74; A61K 39/00; A61K 39/108; A61K 47/69

(52) U.S. Cl. CPC A61K 39/095; A61K 35/74; A61K 39/0258; A61K 47/6911; A61K 2039/6018; A61K 2039/6087; A61K 2039/627

(57) ABSTRACT

The present invention is in the field of conjugating native, non-detergent extracted, outer membrane vesicles (nOMV) to multiple antigens to form multi functionalized nOMV-antigen conjugated derivatives, which are particularly useful for immunogenic compositions and immunisation; processes for the preparation and use of such conjugates are also provided.

### CROSS-REFERENCE TO RELATED APPLICATIONS

This application is a Continuation of U.S. patent application Ser. No. 16/636,560, filed Feb. 4, 2020, which is the National Stage Entry of International Patent Application No. PCT/EP2018/071482, filed Aug. 8, 2018, which claims the priority of GB1712824.0, filed Aug. 10, 2017, all of which are hereby incorporated by reference in their entireties.

This invention is in the field of conjugating “native”, non-detergent extracted, outer membrane vesicles (nOMV) to a series of different antigens, to form multi functionalized nOMV-antigen conjugates, particularly useful for immunisation.

### REFERENCE TO SEQUENCE LISTING

The instant application contains a Sequence Listing which has been submitted electronically in XML file format and is hereby incorporated by reference in its entirety. Said XML copy, created on Apr. 23, 2024, is named VB66368C1-US_23 Apr. 2024_Seq_Listing and is 23,673 bytes in size.

### BACKGROUND ART

Conjugation of antigens to carriers is an established procedure for improving immunogenicity, especially for saccharides. For instance, bacterial capsular saccharides are naturally T-cell independent antigens which give rise to an immune response that lacks several important properties. Conjugation to a carrier moiety converts these saccharides to T-cell dependent antigens which can then produce an immunological memory effect, and also elicit effective immune responses in young children.

One known source of protein carrier in such conjugates is the Outer Membrane Protein Complex (OMPC) from N. meningitidis serogroup B (e.g. see EP-0467714, Merck & co.), which has been included as the carrier in approved H. influenzae B conjugate vaccines. OMPC has also been used as the carrier in protein conjugates. According to the prior art, OMPC is conjugated to an antigen via a protein residue, which may be activated or chemically modified in order to better perform the conjugation with the selected antigen.

Wu et al. (PNAS USA 2006; 103(48): 18243-18248) report that conjugation of Pfs25H (a human malarial transmission-blocking protein) to OMPC resulted in a Pfs25H-OMPC conjugate vaccine that was >1,000 times more potent in generating anti-Pfs25H ELISA reactivity in mice than a similar dose of Pfs25H alone. Conjugation of OMPC to Pfs25H protein can be achieved by reacting maleimide-activated Pfs25H with thiolated outer membrane proteins within OMPC (for a general reference see e.g. WO2006/124712), as shown in Scheme 1.

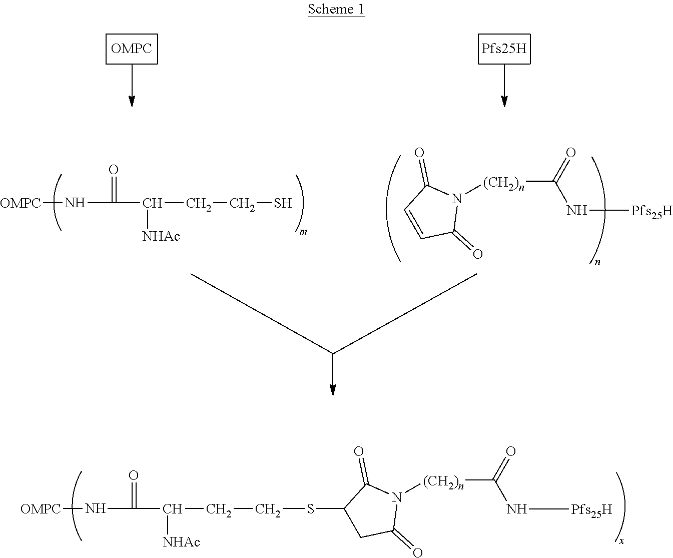

Even if the process can represent a valid synthetic route, the considered vesicles may be difficult to obtain in a pure form, and they are usually collected via laborious processes. Also, the connection with the selected antigen requires the presence and the activation of a suitable vesicle protein, thus posing an additional challenge in light of the use of detergents or chemicals during the vesicle isolation, that can alter the surface proteins composition. Therefore, there is still the need to provide new conjugates useful as immunogenic compounds which overcome the problems of the prior art, and that are achievable by an easy and convenient procedure.

As shown in the above Scheme 1 and according to general procedures of the prior art, the conjugation methods and derivatives thereof contemplate the use of a bivalent heterobifunctional Linker, i.e. a Linker moiety having the terminal ends bearing different functional groups. This is mainly to avoid the cross linking of the vesicle-Linker intermediate with another vesicle particle rather than with the selected antigen. In practice, according to the prior art, the different ends of the Linker are selected depending on the reactive groups on the vesicle and the selected antigen involved in the process, in order to have a selective reaction with the intended part, namely the vesicle on one side and the antigen on the other end side. These methodologies, however, suffer of some drawbacks, mainly related to the selection and functionalization of the Linkers, thus posing some limitation on the choice of the vesicle and antigen to be coupled together. The Applicant has now found that when native, not detergent extracted Outer Membrane Vesicles (nOMVs) are used as starting vesicles it is possible to use a bivalent Linker suitable for the connection with a nOMV surface protein on one side and with a selected antigen on the other end, thus providing a final derivative that still presents the immunogenic activity of both the nOMV and the antigen. Surprisingly, even when the Linker used in the present invention shows identical terminal functional groups, the conjugation of the nOMV with the selected antigen is achieved substantially without the formation of vesicle aggregates or side products, detrimental for the conjugation reaction.

The Applicant has now also found a new and effective method for the multiple functionalization of nOMV, using different chemical approaches that allow the selective conjugation of chosen antigens.

### SUMMARY OF THE INVENTION

In a first aspect, the invention refers to an immunogenic nOMV-antigen conjugate, comprising a native outer membrane vesicle (nOMV) obtained by a detergent free process, having at least a native surface saccharide moiety connected to at least a foreign antigen, and having at least a surface protein residue connected to at least a different foreign antigen through a bivalent Linker.

In a further aspect, the invention refers to a process for preparing said conjugate, comprising the steps of:
i) activating at least a nOMV saccharide moiety, generally bond to the nOMV surface, and
ii) connecting the thus obtained activated nOMV-saccharide to at least one selected antigen, to obtain a nOMV-antigen conjugate;
iii) reacting at least a surface protein residue of the nOMV-antigen conjugate obtained in step ii) with the first terminal portion of a bivalent Linker to obtain a Linker-nOMV-antigen intermediate, and
iv) connecting said Linker-nOMV-antigen intermediate to at least one different antigen via the second terminal portion of the bivalent Linker, thus obtaining the multi-functionalized nOMV derivatives of the invention.

According to the present process, the nOMV-surface bond saccharides are first activated by oxidation, and then reacted with the selected antigen, more preferably under reductive amination conditions. Subsequently, the thus functionalized nOMV is reacted with the linker and then connected through this latter to a different antigen.

The two functionalization mechanisms (via saccharide and via protein/linker as per steps i-ii and iii-iv respectively) can be performed in any order. This means that in an embodiment of the invention the selected nOMV can be first conjugated to an antigen via a nOMV-protein-Linker connection, and subsequently the thus obtained nOMV is conjugated to a different antigen via a surface saccharide moiety. Thus in one embodiment the invention refers to a process for preparing the present conjugates, comprising the steps of:
i) reacting at least a nOMV surface protein residue with the first terminal portion of a bivalent Linker to obtain a nOMV-Linker intermediate, and
ii) connecting said nOMV-Linker intermediate to at least one selected antigen via the second terminal portion of the bivalent Linker, thus obtaining a nOMV-Linker-antigen intermediate,
iii) activating at least a nOMV saccharide moiety of the thus obtained nOMV-Linker-antigen intermediate, and
iv) connecting the thus obtained activated nOMV-saccharide to at least a different antigen, thus obtaining the multi-functionalized nOMV derivatives of the invention.

In an additional aspect, the invention also refers to a conjugate as above set forth, for use as a medicament, particularly as an immunogenic compound, or for the preparation of an immunogenic composition or vaccine.

Still in a further aspect, the invention refers to an immunogenic composition or a vaccine, comprising the above indicated conjugate, and at least one pharmaceutically acceptable carrier or adjuvant; and to a method for raising an immune response in a vertebrate, comprising the administration of said composition or vaccine.

### BRIEF DESCRIPTION OF THE DRAWINGS

FIG. 1 shows a schematic representation of the conjugation of E. coli 405 (FdeC) on S. sonnei nOMV by BS3 chemistry, followed by nOMV oxidation and linkage to E. coli 3526 (SsIE) by reductive amination according to the present invention. Two orthogonal chemistries have been selected according to the invention, to have linkage of 405 to proteins on nOMV and linkage of 3526 on oxidized LPS on nOMV.

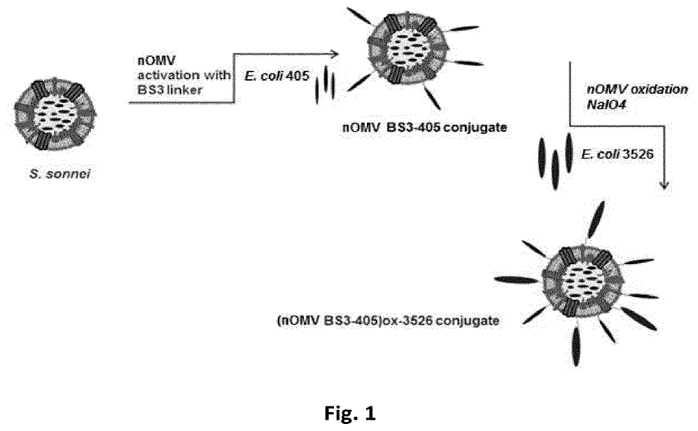

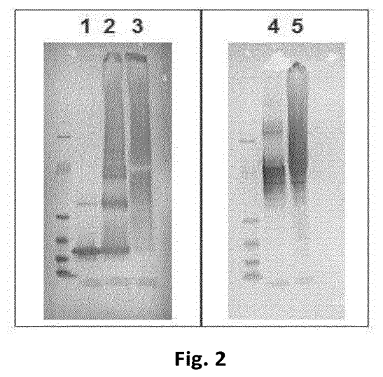

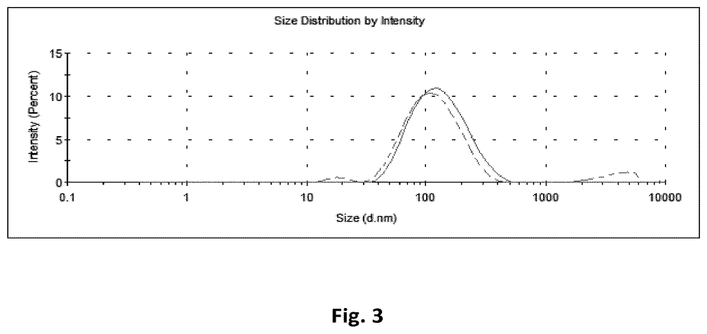

FIG. 2 shows western blot analysis of a GMMA vesicle functionalized with E. coli 405 and E. coli3526 according to the present invention, and as schematically represented in FIG. 1. FIG. 2 confirms the presence of both 405 and 3526 antigens on S. sonnei GMMA, where:
1 E. coli 405 (2 μg), by using anti-405 primary antibody
2 S. sonnei GMMA BS3-405 conjugate (10 μg total protein), by using anti-405 primary antibody 3 (S. sonnei GMMA BS3-405)ox-3526 conjugate (10 μg total protein), by using anti-405 primary antibody
4 E. coli 3526 (2 μg), by using anti-3526 primary antibody
5 (S. sonnei GMMA BS3-405)ox-3526 conjugate (10 μg total protein), by using anti-3526 primary antibody.

FIG. 3: DLS analysis of the nOMV conjugate (dotted line) obtained by first conjugation of GMMA from S. Sonnei, with E. coli 405 via BS3, and subsequently conjugated to E. coli 3526 via polysaccharide oxidation and reductive amination with NaBH₃CN, compared to starting GMMA (continue line) and showing no GMMA aggregation after conjugation.

### DETAILED DESCRIPTION OF THE INVENTION

To facilitate an understanding of the present invention, a number of terms and phrases are defined below. Art-recognized synonyms or alternatives of the following terms and phrases (including past, present, etc. tenses), even if not specifically described, are contemplated.

As used in the present disclosure and claims, the singular forms “a,” “an,” and “the” include plural forms unless the context clearly dictates otherwise; i.e., “a” means “one or more” unless indicated otherwise.

The terms “about” or “approximately” mean roughly, around, or in the regions of. The terms “about” or “approximately” further mean within an acceptable contextual error range for the particular value as determined by one of ordinary skill in the art, which will depend in part on how the value is measured or determined, i.e. the limitations of the measurement system or the degree of precision required for a particular purpose, e.g. the amount of a nutrient within a feeding formulation. When the terms “about” or “approximately” are used in conjunction with a numerical range, it modifies that range by extending the boundaries above and below the numerical values set forth. For example “between about 0.2 and 5.0 mg/ml” means the boundaries of the numerical range extend below 0.2 and above 5.0 so that the particular value in question achieves the same functional result as within the range. For example, “about” and “approximately” can mean within 1 or more than 1 standard deviation as per the practice in the art. Alternatively, “about” and “approximately” can mean a range of up to 20%, preferably up to 10%, more preferably up to 5%, and more preferably up to 1% of a given value.

The term “and/or” as used in a phrase such as “A and/or B” is intended to include “A and B,” “A or B,” A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specified otherwise, all of the designations “A %-B %,” “A-B %,” “A % to B %,” “A to B %,” “A %-B,” “A % to B” are given their ordinary and customary meaning. In some embodiments, these designations are synonyms.

The terms “substantially” or “substantial” mean that the condition described or claimed functions in all important aspects as the standard described. Thus, “substantially free” is meant to encompass conditions that function in all important aspects as free conditions, even if the numerical values indicate the presence of some impurities or substances. “Substantial” generally means a value greater than 90%, preferably greater than 95%, most preferably greater than 99%.

Where particular values are used in the specification and in the claims, unless otherwise stated, the term “substantially” means with an acceptable error range for the particular value.

An “effective amount” means an amount sufficient to cause the referenced effect or outcome. An “effective amount” can be determined empirically and in a routine manner using known techniques in relation to the stated purpose.

As used herein, “heterologous” means the two or more referenced molecules or structures are derived from a different organism. For example, a heterologous antigen is one that is derived from a different organism than the nOMV vesicle to which it is appended. “Homologous” as used herein means the two or more referenced molecules or structures are derived from the same organism.

As used herein, “foreign” means the two or more referenced molecules or structures are not naturally associated with each other. For example, a selected antigen that is herein intended to be “foreign to” a nOMV herein means the antigen is not naturally or innately conjugated to the nOMV molecule even though the antigen and nOMV molecule may originate from the same organism. In this way, a foreign antigen is not necessarily a heterologous antigen but a heterologous antigen is a foreign antigen.

“Sequence identity” can be determined by the Smith-Waterman homology search algorithm as implemented in the MPSRCH program (Oxford Molecular), using an affine gap search with parameters gap open penalty=12 and gap extension penalty=1, but is preferably determined by the Needleman-Wunsch global alignment algorithm (see e.g. Rubin (2000) Pediatric. Clin. North Am. 47:269-285), using default parameters (e.g. with Gap opening penalty=10.0, and with Gap extension penalty=0.5, using the EBLOSUM62 scoring matrix). This algorithm is conveniently implemented in the needle tool in the EMBOSS package. Where the application refers to sequence identity to a particular SEQ ID, the identity is intended to be calculated over the entire length of that SEQ ID.

The term “w/w %” indicates the weight percentage of a given component, over a different component or over the whole content of a composition, as indicated.

Analogously, the term “% v/v” indicates the volume percentage of a given component, over a different component or over the whole content of a composition, as indicated.

The term “—OAg” (0-antigen) is used within the present invention to indicate an antigen functionality present in the lipopolysaccharides (LPS) or lipooligosaccharides (LOS) on the surface of the considered nOMV, useful for the conjugation with a proper antigen (generally indicated as Ag) according to the invention. In more details, the LPS are generally formed by three different portions, known as: lipidA (responsible for the toxicity of LPS), core oligosaccharide and the—OAg chain, a repetitive glycan polymer and major contributor to the serological specificity of bacteria.

The term “bivalent homobifunctional Linker” or “homologous Linker” indicates a linking unit presenting two terminal ends bearing the same functional group, and able to react with the nOMV protein on one side, and with the selected antigen on the other side, where nOMV protein and selected antigen are as herein below described in details.

Similarly, the term “bivalent heterobifunctional Linker” or “heterologous Linker” indicates a linking unit presenting two terminal ends bearing different functional groups, and able to specifically react with the nOMV protein on one side, and with the selected antigen on the other side, where nOMV protein and selected antigen are as herein below described in details.

The term “linear or branched C1-Cₓ alkyl or alkenyl group” comprises in its meaning a bivalent satured or unsatured linear or branched alky or alkenyl group having 1 to x carbon atoms. For instance, the term bivalent C₁-C₁₀ alkyl or alkenyl group comprises in its meaning a bivalent satured or unsatured alky or alkenyl group having 1 to 10 carbon atoms such as methyl, ethyl, vinyl, allyl and the like.

As herein used, the term “saccharide (or sugar) moiety” comprises in its meaning mono saccharides, as well as polysaccharide units. It will be appreciated that saccharide moieties can exist in open and closed (ring) form and that, while closed forms are shown in structural formulae herein, open forms are also encompassed by the invention. Similarly, it will be appreciated that saccharide moieties can exist in pyranose and furanose forms and that, while pyranose forms are shown in structural formulae herein, furanose forms are also encompassed. Different anomeric forms of saccharide moieties are also encompassed.

The term “oligosaccharide” comprises in its meaning polysaccharides having from 3 to 10 monosaccharide units.

Unless otherwise provided, the term “polypeptide” refers to polypeptides of any length capable to act as a selected antigen. The amino acid polymer forming the polypeptide of the invention, may be linear or branched, it may comprise modified amino acids, and it may be interrupted by non-amino acids. The term also encompasses an amino acid polymer that has been modified naturally or by intervention; for example, disulphide bond formation, glycosylation, lipidation, acetylation, phosphorylation, or any other manipulation or modification, such as conjugation with a labelling component. Also included within the definition are, for example, polypeptides containing one or more analogues of an amino acid (including, for example, unnatural amino acids, etc.), as well as other modifications known in the art. Polypeptides can occur as single chains or associated chains.

“Average molecular weight” is intended to indicate the average molecular weight obtained by the ordinary arithmetic mean or average of the molecular masses of the individual component, e.g. amino acids in case of polypeptide derivatives.

The term “capsular polysaccharides/saccharides” (CPSs) indicates those saccharides which can be found in the layer that lies outside the cell envelope of bacteria, thus being part of the outer envelope of the bacterial cell itself. CPSs are expressed on the outermost surface of a wide range of bacteria, and in some cases even in fungi.

Unless otherwise provided, the term “conjugation” indicates the connection or linkage of the subjected entities, particularly referred to the nOMV and the selected antigen moieties.

By “immunologically effective amount”, it is meant that the administration of that amount to an individual, either in a single dose or as part of a series, is effective for treatment or prevention.

This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

The term “nOMV” herein indicates vesicle isolated from the medium or sheared from cells, and they are intact membrane vesicles not exposed to detergents or denaturing agents, i.e. not detergent extracted. The nOMVs of the invention present the outer membrane proteins (OMP) and lipopolysaccharide (LPS) in their native conformation and correct orientation in the natural membrane environment, and usually lack the cytoplasmatic components.

On the contrary, the term “OMV” or “dOMV” encompasses a variety of proteoliposomic vesicles obtained by disruption of the outer membrane of a Gram-negative bacterium typically by a detergent extraction process to form vesicles therefrom. Outer membrane protein complexes (e.g.

OMPC from Neisseria meningitidis) may be considered in such definition, since having three dimensional structure and composition similar to dOMV, and being isolated via detergent extraction procedures (see e.g. EP0467714, U.S. Pat. Nos. 4,271,147, 4,459,286 and 4,830,852). The detergent extraction process removes LPS and phospholipids, together with immunoprotective lipoproteins. Such removal changes the native vesicle structure and promotes aggregation. The aggregation may lead to consequent issues in terms of process development (yield, consistency of production and stability). Differently from nOMVs, characterized by defined homogeneous size distribution (typically in the range 20-250 nm, measured by Dynamic Light Scattering DLS technique), the dOMVs have an undefined heterogeneous size distribution (usually in the range 550-5500 nm as measured by Dynamic Light Scattering DLS technique) caused by detergent-induced vesicle aggregation (see for a general reference, Vaccine 28, 2010, 4810). The detergent extraction process also causes contamination of OMV containing composition (e.g. vaccines) with cytoplasmic proteins as a result of bacterial cell lysis.

According to prior art methodologies, dOMVs and nOMVs may be analysed and described in terms of size, shape and overall appearance of impurities or contaminating non-OMV materials (like vesicle aggregates or detergent residues in case of dOMVs) using the Transmission Electron Microscopy (TEM). For detailed references regarding the differences between dOMVs and nOMVs see e.g. van de Waterbeemd (2013) J. Prot. Res. “Quantitative Proteomics Reveals Distinct Differences in the Protein Content of Outer Membrane Vesicle Vaccines”; and J. Klimentova et al. Microbiological Research 170 (2015) 1-9 “Methods of isolation and purification of the outer membrane vesicles from gram-negative bacteria”.

As herein used, unless otherwise provided the term “connection” or “conjugation” means the formation of a covalent bond between the interested entities or moieties.

As above mentioned, the present conjugates are obtained by connecting at least one nOMV surface saccharide moiety to a selected foreign antigen and by covalently connecting at least a protein unit on the same nOMV to a different foreign antigen, via a suitable bivalent Linker.

In this respect, “foreign antigens”, means that they do not form part of the vesicle, and typically the antigen connected through saccharide residue is different from the one connected to the surface protein via a bivalent linker. Furthermore, as well as being capable of inducing an immune response against the coupled antigens, the conjugates of the invention are also capable of inducing an immune response against the nOMV component itself, differently from dOMV-antigen conjugates of the art where the immune activity relies mainly on the antigen portion and not on the detergent extracted vesicle. On the contrary, the conjugation of different antigens to the same nOMV according to the present invention does not significantly impact on the ability of the nOMV to induce its own immune response. Of note, when nOMV and the selected foreign antigens are from different sources, the conjugates of the invention may be useful for the preparation of immunogenic compositions or vaccines based on both the nOMV and the conjugated antigens activity. Even further, the possibility to conjugate more than 2 foreign antigens to the same nOMV according to the selective functionalization of the invention is potentially useful for the preparation of a wide range of multivalent nOMV conjugates, endowed with a tailored immunogenic activity.

The nOMVs in accordance with the present invention are collected and isolated substantially without the use of detergents, differently for instance from dOMVs of the prior art obtained via a deoxycholate extraction or using zwitterionic detergents like Empigen BB (see e.g. U.S. Pat. No. 4,707,543) or similar. On the contrary, it has to be highlighted that a detergent extraction step may be undesirable in the present invention, for a series of reasons, among which the fact that a detergent would reduce the amount of lipopolysaccharide (LPS)/lipooligosaccharide (LOS) present on the vesicle, which can be indeed useful for the conjugation with the selected antigen as herein below described. The conjugates of the invention offer several advantages compared to unconjugated antigens; for example their ability to act as a multivalent vaccine as discussed above, and their improved immunogenicity over unconjugated antigens. In addition, the conjugates of the invention have several advantages over the vesicle-protein conjugates that have been used to date. Firstly, the nOMVs conjugates can be prepared with fewer steps compared to dOMV conjugates in which antigens were coupled to dOMV proteins, and in particular without requiring the expensive and time consuming step of protein derivatisation. Secondly, the production of nOMVs can be more reliable and convenient than the preparation of dOMVs by detergent extraction. Even further, unreacted antigens from the conjugation mixture can be recycled for use in a further conjugation step, improving the efficiency of production of the conjugates.

In further details, the nOMVs are naturally-occurring membrane vesicles that form spontaneously during bacterial growth and are released into culture medium. They can be obtained e.g. by culturing bacteria in broth culture medium, separating whole cells from the smaller nOMVs in the broth culture medium (e.g. by filtration or by low-speed centrifugation to pellet only the cells and not the smaller vesicles), and then collecting the nOMVs from the cell-depleted medium (e.g. by filtration, by differential precipitation or aggregation, by high-speed centrifugation to pellet the vesicles). Strains for use in production of nOMVs can generally be selected on the basis of the amount of nOMVs produced in culture. The present nOMVs are characterised by the fact of being collected and isolated following a detergent-free procedure. Preferably, the present nOMVs are released into the fermentation broth and are purified using a centrifugation and subsequent filtration step (for a general reference see e.g. Clin Vaccine Immunol. 2016 April; 23(4): 304-314).

Still preferably, the present nOMVs are released into the fermentation broth and are purified using the following two consecutive Tangential Flow Filtration (TFF) steps: (i) a microfiltration in which the culture supernatant containing the nOMV is separated from the bacteria, and (ii) an ultrafiltration in which the nOMV are separated from soluble proteins (for a general reference see e.g. PLoS One. 2015; 10(8): e0134478). The thus obtained nOMVs can then directly be used within the present invention without additional purification/isolation steps. The presently considered nOMVs have a preferred size distribution comprised from 20 to 250 nm, measured by Dynamic Light Scattering DLS technique.

According to some embodiments, the nOMVs are prepared from wild-type bacteria or from bacteria which have been genetically manipulated generally to increase immunogenicity (e.g. to hyper-express immunogens), to reduce toxicity, to inhibit capsular saccharide synthesis, to down-regulate immunodominant antigen expression, and the like. They also may be prepared from hyperblebbing strains. The nOMVs of the invention may also express exogenous proteins on their surface and they may be endotoxin-depleted.

Preferably, the nOMVs suitable for the invention are produced from genetically-modified bacterial strains that are mutated to enhance vesicle production, and optionally also to remove or modify antigens (e.g. lipid A) and/or to over-express homologous antigens or antigens from other organisms. Said preferred nOMVs are also known as Generalized Modules of Membrane Antigens (GMMA) as e.g. described in PLoS One. 2015; 10(8): e0134478.

Enhanced spontaneous generation of vesicles can be achieved, for example, by targeted deletion of proteins involved in maintenance of membrane integrity. It has been observed that the outer surface of nOMVs substantially corresponds to the outer surface of the bacterium from which they are derived, preserving the membrane antigens (including e.g. lipopolysaccharides, lipooligosaccharides and lipoproteins) in the context of the membrane. Advantageously, the nOMVs used in the invention (unlike detergent-extracted dOMVs) retain these outer membrane components in their native conformation and correct orientation, better preserving immunogenicity against the bacterial strain from which they are derived.

Generally, the nOMVs suitable for the invention may be prepared from any suitable bacterium, where preferred bacteria include, but are not limited to: Neisseria (e.g. in particular N. meningitidis of any serogroups including A, B, C, X, Y or W135, or from a non-pathogenic Neisseria), Shigella (such as S. sonnei, S. flexneri, dysenteriae or boydii), Salmonella enterica serovars (such as Paratyphi A, B or C, Enteritidis, Typhi or Typhimurium), Haemophilus influenzae (e.g. non-typable H. influenzae), Vibrio cholerae, Bordetella pertussis, Mycobacterium smegmatis, Mycobacterium bovis BCG, Escherichia coli, Bacteroides (including Porphyromonas), Pseudomonas aeruginosa, Helicobacter pylori, Brucella melitensis Campylobacter jejuni, Actinobacillus actinomycetemcomitans, Xenorhabdus nematophilus, Moraxella catarrhalis, or Borrelia burgdorferi.

Particularly preferred bacteria are selected from at least one of: S. sonnei, S. flexneri, Salmonella bacterium, and meningococcus, particularly meningococcus serogroup B.

Virulent Shigella strains possess a 220 kb plasmid that mediates virulence properties. This “virulence plasmid” has been shown to encode the genes for several aspects of Shigella virulence, including adhesins for target epithelial cells, the invasion plasmid antigens, virF, virG, and the like. A Shigella used with the invention may or may not possess a virulence plasmid. Absence of the plasmid can stabilise the strain during industrial culture, attenuate the strain by removing virulence factors (thereby increasing safety of manufacture), avoid the presence of the ShET-2 enterotoxin (encoded by the ospD3 or sen gene on the plasmid), and avoid the presence of msbB2 which is a second copy of the msbB gene responsible for acylation of lipid A. Absence of the virulence plasmid may also disrupt the lipopolysaccharide. However, the biosynthesis genes for the—OAg should preferably be retained, either by maintenance of a mutated virulence plasmid, or by inclusion in a further plasmid or cloning into the bacterial chromosome.

As far as Salmonella bacterium is concerned, a particularly preferred strain is selected from: Salmonella Typhimurium, Salmonella Enteritidis and Salmonella Paratyphi A.

Meningococcus bacteria nOMVs are also preferred. Such vesicles can be prepared from any meningococcal strain. The vesicles are preferably prepared from a serogroup B strain, but it is also preferred to prepare them from serogroups other than B, such as one of: A, C, W135 or Y, according to procedures known in the art. The strain may be of any serotype (e.g. 1, 2a, 2b, 4, 14, 15, 16, etc.), any serosubtype (e.g. P1.4), and any immunotype (e.g. L1; L2; L3; L3,7; L3,7,9; L10; etc.). The meningococci may be from any suitable lineage, including hyperinvasive and hypervirulent lineages, preferably any of the following seven hypervirulent lineages: subgroup I; subgroup III; subgroup IV-1; ET-5 complex; ET-37 complex; A4 cluster; lineage 3. Most preferably, OMVs are prepared from the strain NZ98/254, or another strain with the P1.4 PorA serosubtype.

In another embodiment, bacteria for preparing nOMVs suitable for the invention may be mutant strains which have been manipulated e.g. to enhance vesicle production, to express one or more desired antigen(s), and/or to knockout or modify an undesired gene (e.g. one which encodes a toxin or which encodes an enzyme involved in generating a toxic product, such as endotoxin).

In this direction, other preferred nOMVs suitable for the invention are produced by a Salmonella bacterium, particularly a S. Typhimurium (also known as Salmonella enterica serovar Typhimurium) which does not express a functional ToIR protein.

Where the vesicles are prepared from E. coli, Shigella or Salmonella the bacterium may express no more than 4 of ToIA, ToIB, ToIQ, ToIR and Pal proteins. Thus at least one protein from the natural five-protein Tol-Pal system may be absent, resulting in a bacterium which, during growth in culture medium, releases greater quantities of outer membrane vesicles into the medium than the same bacterium expressing all 5 Tol-Pal proteins. Preferably ToIR is not expressed, but the other four proteins may be expressed (i.e. a AToIR strain).

In preferred embodiments, at least one of the five Tol-Pal proteins in E. coli, Shigella or Salmonella is removed e.g. by deletion or inactivation of the gene encoding the protein. Thus the bacterium may express 0, 1, 2, 3 or 4 of ToIA, ToIB, ToIQ, ToIR and Pal proteins. Removal of one of the five proteins can suffice, in which case the bacterium expresses only 4 of these proteins. Preferably the ToIR protein is removed e.g. by inactivation of a starting strain's tolR gene. Thus a preferred bacterium may be tolA+ tolB+ tolQ+ ToIR-Pal+.

In some embodiments, the bacterium expresses all five Tol-Pal proteins, but at least one is mutated to cause hyperblebbing. For instance, the ToIA, ToIQ, ToIR and/or Pal protein may be mutated such that the protein retains its membrane localisation but its interactions with other members of the Tol-Pal system are disrupted. The bacterium will thus retain ToIA, ToIQ and ToIR as transmembrane proteins in the inner membrane, and Pal protein as a periplasm-facing lipoprotein in the outer membrane, but at least one of the ToIA, ToIQ, ToIR and/or Pal proteins is mutated and not fully functional.

In addition other mutations may also be present e.g. to give OAg-deficient strains, for instance in those cases where the—OAg functionality is not intended for desired immune response, or in those cases where the—OAg may negatively impact the immunogenicity against the heterologous antigen. In this direction, possible mutations may be AgalU, AgalE or AwbaP in E. coli, Shigella or Salmonella strains.

In one further preferred embodiment, a meningococcus does not express a functional MItA protein. Knockout of MItA (the membrane-bound lytic transglycosylase, also known as GNA33) in meningococcus provides bacteria which spontaneously release large amounts of nOMVs into culture medium, from which they can be readily purified. For instance, the vesicles can be purified using the two stage size filtration process, comprising: (i) a first filtration step in which vesicles are separated from the bacteria based on their different sizes, with the vesicles passing into the filtrate; and (ii) a second filtration step in which the vesicles are retained in the retentate.

In the present invention, it is preferred that—OAg is present on the nOMVs because it has been observed (e.g. nOMVs from Salmonella and Shigella) that, the presence of the—OAg on the surface of said nOMVs is advantageous in providing a multivalent vaccine, as the—OAg can act as a protective antigen. Some preferred strains have penta- or tetra-acylated less toxic LPS, which includes attached —OAg, after the mutation of msbB, htrB, ddg and/or PagP (see e.g. Rossi O et al, Clin Vaccine Immunol. 2016 Apr. 4; 23(4):304-14 and Rossi O et al, J Biol Chem. 2014 Sep. 5; 289(36):24922-35.

In Neisseria, the strain has preferably a modified fur gene. According to this embodiment, mutant Neisseria are engineered to reduce or switch off expression of at least one gene involved in rendering toxic the lipid A portion of LPS, in particular of Ipxl1 gene. In this way, the resulting nOMVs present a reduced toxicity respect to the wild type strain, since the conversion of acylated lipid A in a less acylated form.

Similarly, preferred mutant Neisseria for the invention are engineered to reduce or switch off expression of at least one gene involved in the capsular saccharide synthesis or export, in particular of synX and/or ctrA genes. In this way, the resulting nOMVs may present a cross protection versus different serotypes, particularly appreciated by the skilled in the art.

In preferred embodiments a strain may include one or more of the knockout and/or hyper-expression mutations disclosed e.g. in Fukusawa et al. (1999), Vaccine 17:2951-2958. For instance, following the therein guidance and nomenclature, useful genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB; (b) CtrA, CtrB, CtrC, CtrD, FrpB, GalE, HtrB/MsbB, LbpA, LbpB, LpxK, Opa, Opc, PhoP, PilC, PmrE, PmrF, SiaA, SiaB, SiaC, SiaD, TbpA, and/or TbpB; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB; or (d) CtrA, CtrB, CtrD, FrpB, OpA, OpC, PilC, PorB, SiaD, SynA, SynB, SynX and/or SynC.

As far as the nOMV saccharide moiety is concerned, it has to be noted that it can be part of the—OAg functionality naturally present on the surface of the nOMV (e.g. in LPS or LOS), or it can be present within a different nOMV surface portion, e.g. a CPS, as herein below described in details. Advantageously, any proper antigen may be conjugated to the nOMV to obtain the nOMV-antigen conjugates of the invention, preferably in the form of a (poly)saccharide or polypeptide.

In any case, the connection of one or more selected antigens produces an immunogenic conjugate which can raise an immune response which recognises said antigens, and which also recognises one or more components in the nOMV, thereby conveniently providing a multivalent vaccine. Generally, antigens will be included in the present conjugates at a concentration which is high enough to elicit, when administered to a host, an immune response which recognises that antigen.

As far as the functionalization of the nOMV through a surface protein is concerned, the present invention surprisingly shows that a homobifunctional Linker can be used in the preparation of nOMV-antigen conjugates, without incurring in the prior art problems related e.g. to vesicles cross reaction or aggregation. In practice, the nOMV is functionalized with the homobifunctional Linker by reacting proper functional groups of at least one nOMV surface protein with one end of the Linker. By that, a nOMV-Linker intermediate is covalently formed, still having the other end of the Linker available for the subsequent reaction with the selected antigen. Thus, the second end of the Linker will react with the selected antigen, in a specific and selective way, leading to the final nOMV-Linker-antigen derivative, and substantially avoiding intermediate cross reactions or aggregations. This is particularly valuable since the selective functionalization can be performed on a nOMV already conjugated to a first different antigen, or according to another embodiment of the invention, the firstly obtained nOMV-Linker-Antigen intermediate can undergo a second selective conjugation to a different antigen, via a nOMV polysaccharide, to give the conjugates of the invention. As indicated in the present experimental part, the same functionalization when carried out considering a dOMV as starting vesicle leads to the formation of vesicle-Linker-vesicle aggregates, which are not suitable for a subsequent reaction with the selected antigen.

Surprisingly it has now found that not only the use of nOMV as starting vesicle can overcome the aggregation problems of the prior art, but also it is now possible to use a variety of bifunctional Linkers that lead to the preparation of a series of nOMV-antigens derivatives according to the invention, retaining the immunogenic profile of the conjugated components. Of note, the functionalization of the nOMV through the surface protein as just set forth can be performed before or after the functionalization of the nOMV with a different antigen through a saccharide moiety, providing in any case effective and useful multi-functionalized nOMV derivatives.

The nOMVs are covalently linked to the bifunctional Linker by way of at least one protein residue, generally located on the surface of the vesicle. In this direction, the proteins will preferably react with a terminal end of the Linker by means of one or more amino, thiol or hydroxyl amino acid functionalities, being this latter an alpha hydroxyl group or part of the carboxy aminoacid functionality. Preferably, the protein functional group is an amino group, more preferably a primary amine (—NH₂). These functional groups may naturally be present in the amino acid portions of interest, or even introduced artificially for the purposes of conjugation. When the selected Linker is a homobifunctional bivalent Linker, it will be understood that the proteins functional group and the antigen functional group that will react with the terminal portion of the Linker will be preferably the same. By way of example, lysine aminoacid residues of one or more nOMV proteins will react with the Linker (e.g. BS3) via the corresponding —NH₂ functional group. In the same way, also a selected antigen (e.g. E. Coli 405) will react with the remaining free terminal portion of the Linker via the relevant amino (—NH₂) groups. Of note, and as well explained in the present description, the reaction occurs without substantial cross reaction or aggregation formation, thus leading to the final product, useful for the preparation of multivalent vaccines, in a very reliable and versatile way, and differently from using dOMVs, as indicated in the comparative Examples herein provided. Preferred amino acid residues include, but are not limited to: arginine, lysine, asparagine, glutamine, aspartic or glutamic acid, cysteine and histidine. Preferably, the nOMV proteins are those having one or more aminoacid moiety showing free amino groups, preferably primary —NH₂ groups. Even more preferably said aminoacid moiety is the arginine and/or lysine, whereby different —NH₂ groups form different arginine and/or lysine proteins are able to selectively react with the linker according to the present invention.

As far as the bivalent Linker is concerned, this is typically a molecule of a certain length, with a suitable water solubility and polarity able to covalently bind the nOMV proteins and the antigen by its terminal ends respectively. In order to optimize the solubility of the chosen Linker, it may be expedient to introduce one or more polar group such as sulfate, sulfite, phosphate and the like, or even use the corresponding salt thereof, e.g. as alkaline or alkaline earth metal salts, where possible. Due to the versatility of the present invention, it is possible to use different Linkers, in terms e.g. of lengths, polarity, and steric hindrance, thus providing a covalent bond with both the nOMV protein residues and the selected antigen. By that, the invention allows the preparation of a variety of nOMV-Linker-antigens conjugates, endowed with remarkable and specific behavior, with particular regard to their immunogenicity and activity.

The Linker can be heterobifunctional (i.e. bearing two different terminal functionalities) or, preferably homobifunctional (i.e. having equal terminal functionalities). Even more preferably, the Liker is symmetric with respect to a hypothetical vertical axis.

Thus the bivalent Linker according to the present invention has a general formula (I):

X-L-X′ (I)

wherein:

X and X′ are different to each other or the same, and are a functional group able to selectively react with nOMV proteins on one hand and with the selected antigen on the other hand, preferably by forming ester, amido or thioester moieties;

-L- is a bivalent linear or branched C₁-C₁₅ alkyl or alkenyl group optionally substituted, and optionally interrupted by one or more heteroatom selected from: oxygen (—O—), sulfur (—S—), nitrogen (—NH— or optionally substituted —N— group) and the like.

In one embodiment, -L- is preferably a bivalent linear C₃-C₁₂ alkyl group, optionally substituted or interrupted by one or more oxygen (—O—) heteroatom. In a still more preferred embodiment, -L- is a bivalent linear C₃-C₆ alkyl group. According to formula (I), the Linker is further characterized by having both terminal portions bearing two functionalities X and X′ which are preferably the same thus providing a bivalent homo-functional Linker. In one embodiment, the X and/or X groups can be any which form esters, thioester or amide when combined with a hydroxyl, thiol or amino functionality respectively.

Preferably, X and/or X′ are N-hydroxysuccinimide ester derivatives, more preferably selected from at least one of:

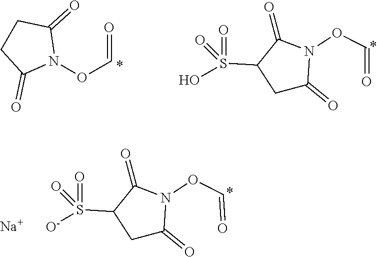 [SMILES: O=CON1C(=O)CCC1=O ; O=CON1C(=O)CC(S(=O)(=O)O)C1=O ; [Na+] ; O=CON1C(=O)CC(S(=O)(=O)[O-])C1=O]

wherein the * represents the point of contact with the -L- spacer in formula (I), as above defined. Thus, in a still preferred embodiment, the Linker is selected from at least one of: disuccinimidyl glutarate (DSG), disuccinimidyl suberate (DSS), succinimidyl 3-(2-pyridyldithio)propionate (SPDP), Succinimidyl 6-(3-[2-pyridyldithio]-propionamido)hexanoate (LC-SPDP), sulfosuccinimidyl 6-(3′-(2-pyridyldithio)propionamido)hexanoate (sulfo-LC-SPDP), 4-succinimidyloxycarbonyl-α-methyl-α-(2-pyridyldithio)toluene (SMPT), sulfosuccinimidyl-6-[α-methyl-α-(2-pyridyldithio)tolueamideo]hexanoate (sulfo-LC-SMPT), succinimidyl 4-(N-maleimidomethyl)cyclohexane-1-carboxylate (SMCC), sulfosuccinimidyl 4-(N-maleimidomethyl)cyclohexane-1-carboxylate (suflo-SMCC), m-maleimidobenzoyl-N-hydroxysuccinimide ester (MBS), m-maleimidobenzoyl-N-hydroxysulfosuccinimide ester (sulfo-MBS), N-succinimidyl (4-iodoacetyl)aminobenzoate (SIAB), sulfosuccinimidyl (4-iodoacetyl)aminobenzoate (sulfo-SIAB), succinimidyl 4-(N-maleimidophenyl)butyrate (SMPB), sulfosuccinimidyl 4-(N-maleimidophenyl)butyrate(sulfo-SMPB), N-γ-maleimidobutyryl-oxysuccinimide ester (GMBS), N-γ-maleimidobutyryl-oxysulfosuccinimide ester (sulfo-GMBS), succinimidyl-6-((((4-(iodoacetyl)amino)methyl)cyclohexane-1-carbonyl)amino)hexanoate (SIACX), succinimidyl 6[6-(((iodoacetyl)amino)hexanoyl)amino]hexanoate (SIAXX), succinimidyl-4-(((iodoacetyl)amino)methyl)cyclohexane-1-carboxylate (SIAC), succinimidyl 6-[(iodoacetyl)amino]hexanoate (SIAX) and p-nitrophenyl iodoacetate (NPIA), N-hydroxysuccinimide, N oxysuccinimide and adipic acid N-hydroxysuccinimide diester (SIDEA) and Bis(sulfosuccinimidyl) suberate (BS3, CAS No. 82436-77-9).

In one embodiment, additional preferred bifunctional Linkers reactive with amines for use with the invention are selected from at least one of: acryloyl halides (e.g. chloride), ethylene glycol bis[succinimidylsuccinate], bis(sulfosuccinimidyl)tri(ethylene glycol) (BS(PEG)3), bis(sulfosuccinimidyl)tetra(ethylene glycol) (BS(PEG)4), bis(sulfosuccinimidyl)penta(ethylene glycol) (BS(PEG)5) and bis(sulfosuccinimidyl)exa(ethylene glycol) (BS(PEG)6), where bis(sulfosuccinimidyl)penta(ethylene glycol) (BS(PEG)5, CAS No 756526-03-1) is particularly preferred.

Preferred homobifunctional Linkers able to react with thiol functional groups on nOMV protein and antigen according to the invention, are those having X and/or X′ selected from at least one of: 2-pyridyldithio, maleimide or iodoacetyl residue.

Other Linkers suitable for the reaction with the nOMV protein hydroxyl group as above defined, are selected from at least one of: Adipic acid dihydrazide (ADH), β-propionamido, nitrophenyl-ethylamine, haloacyl halides, 6-aminocaproic acid.

Among the Linkers useful within the present invention, (BS(PEG)5), Disuccinimidyl glutarate (DSG) or a salt thereof, and BS3 are preferred ones, being BS3 even more preferred (for a general reference on BS3 see e.g. U.S. Pat. No. 4,965,338). According to a still preferred embodiment, DSG is particularly useful when operating at pH of about 9. Surprisingly, the efficacy of the conjugation reaction can be conveniently increased when (BS(PEG)5) or BS3 are used as bivalent Linker, substantially in the absence of vesicle aggregates formation. In this respect, it has to be highlighted that the use of BS3 according to the present invention does not provide substantial crosslinking of nOMV surface proteins to form high-molecular-mass aggregates, but rather, selective reaction with nOMV on one terminal end, and with the selected antigen on the other end. This behavior is further supported by the herein enclosed experimental part, where example 4 (comparative, using dOMV) is described.

In one embodiment of the invention, the nOMV is conjugated to at least one homologous antigen, i.e. derived from the same organism from which the nOMVs are derived. In a still preferred embodiment, the nOMV is conjugated to at least one heterologous antigen i.e. derived from a different organism from the organism from which the nOMVs are derived. In any case, the antigens may generally be selected from any immunogenic polypeptides, i.e. polypeptides able to elicit an immune response when administered to a subject. Polypeptides used with the invention will include an amino acid having a residue, or a side chain, with a functional group suitable for conjugation, preferably an amino or a thiol group, even more preferably of general formula: —NH₂ or —SH. These residues may naturally be present in an antigen, or they may be introduced artificially for the purposes of conjugation. Preferred amino acid residues include, but are not limited to: arginine, lysine, asparagine, glutamine, cysteine and histidine. The most preferred amino acid residue for conjugation is lysine.

Polypeptide antigens suitable for the invention are preferably prepared in substantially pure or substantially isolated form (i.e. substantially free from other polypeptides). They can be prepared by various means e.g. by chemical synthesis (at least in part), by digesting longer polypeptides using proteases, by translation from RNA, by purification from cell culture (e.g. from recombinant expression or from native culture), and the like. Recombinant expression in an E. coli host is a useful expression route. Polypeptide antigens can take various forms (e.g. native, fusions, glycosylated, non-glycosylated, lipidated, disulfide bridges and the like).

Polypeptide antigens suitable for the invention have a preferred average molecular weight of at least 1 kDa, more preferably of at least 3.5 kDa, even more preferably from 10 to 180 kDa, still more preferably, the average molecular weight is comprised from 15 to 75 kDa.

Further preferred polypeptide antigens for conjugating to nOMVs according to the present invention comprise an epitope from a fungal, bacterial, protozoan or viral polypeptide. Preferred protozoan polypeptides are from a Plasmodium (such as P.falciparum, P.vivax, P.ovale).

Particularly preferred bacterial polypeptides are selected from: E. coli, N.meningitidis, and Streptococci (such as S.agalactiae, S.pneumoniae, S.pyogenes).

Preferred E. coli polypeptide antigens include CTF1232, 405 (FdeC) and 3526 (SsIE). As a non-limiting preferred example, nOMV from Shige//a can be conjugated to 405 and 3526, according to the present invention, to generate a multivalent vaccine covering both E. coli and Shige//a. In one embodiment, the considered N. meningitidis polypeptides are able, when administered to a mammal, to elicit an antibody response that is bactericidal against meningococcus. Preferred N. meningitidis polypeptides for use with the invention are selected from at least one of: NHBA, NadA, NsPA, NhhA, App and fHbp, as herein below detailed.

E. coli 3526 (SsIE) The E. coli 3526 (SsIE) antigen was included in the published genome sequence for E. coli strain IHE3034 as gene ECOK1_3385 (GenBank accession number CP001969; SEQ ID NO: 15 herein). The sequences of 3526 (SsIE) antigen from many strains have been published since then. Various immunogenic fragments of the antigen have also been reported. Preferred 3526 (SsIE) antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 15; and/or (b) comprising a fragment of at least ‘n’ consecutive amino acids of SEQ ID NO: 15, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 15. The most useful 3526 (SsIE) antigens of the invention can elicit antibodies which, after administration to a subject, can bind to a polypeptide consisting of amino acid sequence SEQ ID NO: 15. Advantageous 3526 (SsIE) antigens for use with the invention can elicit functional antibodies after administration to a subject.

E. coli 405 (FdeC) The E. coli 405 (FdeC) antigen was included in the published genome sequence for E. coli strain IHE3034 as gene ECOK1_0290 (GenBank accession number CP001969;). The sequences of 405 (FdeC) antigen from many strains have been published since then. Various immunogenic fragments of the antigen have also been reported. Preferred 405 (FdeC) antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) of the sequences of 405 (FdeC) antigen, more preferably as indicated in the herein Seq ID No. 16; and/or (b) comprising a fragment of at least ‘n’ consecutive amino acids of 405 (FdeC) antigen, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 16. The most useful 405 (FdeC) antigens of the invention can elicit antibodies which, after administration to a subject, can bind to 405 antigen. Advantageous 405 (FdeC) antigens for use with the invention can elicit functional antibodies after administration to a subject.

### Nhba Antigen.

The NHBA antigen was included in the published genome sequence for meningococcal serogroup B strain MC58 as gene NMB2132 (GenBank accession number GI:7227388; SEQ ID NO: 2 herein). The sequences of NHBA antigen from many strains have been published since then. Various immunogenic fragments of the NHBA antigen have also been reported. Preferred NHBA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) comprising a fragment of at least ‘n’ consecutive amino acids of SEQ ID NO: 2, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 2. The most useful NHBA antigens of the invention can elicit antibodies which, after administration to a subject, can bind to a meningococcal polypeptide consisting of amino acid sequence SEQ ID NO: 2. Advantageous NHBA antigens for use with the invention can elicit bactericidal anti-meningococcal antibodies after administration to a subject.

### Nada Antiqen.

The NadA antigen was included in the published genome sequence for meningococcal serogroup B strain MC58 (see e.g. Tettelin et al. (2000) Science 287:1809-1815) as gene NMB1994 (GenBank accession number GI:7227256; SEQ ID NO: 3 herein). The sequences of NadA antigen from many strains have been published since then, and the protein's activity as a Neisserial adhesin has been well documented. Various immunogenic fragments of NadA have also been reported. Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) comprising a fragment of at least ‘n’ consecutive amino acids of SEQ ID NO: 3, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 3. The most preferred NadA antigens of the invention can elicit antibodies which, after administration to a subject, can bind to a meningococcal polypeptide consisting of amino acid sequence SEQ ID NO: 3.

Advantageous NadA antigens for use with the invention can elicit bactericidal anti-meningococcal antibodies after administration to a subject. SEQ ID NO: 7 is one such fragment.

### Nspa Antigen.

The NspA antigen was included in the published genome sequence for meningococcal serogroup B strain MC58 (see e.g. Tettelin et al. (2000) Science 287:1809-1815) as gene NMB0663 (GenBank accession number GI:7225888; SEQ ID NO: 4 herein). The sequences of NspA antigen from many strains have been published since then. Various immunogenic fragments of NspA have also been reported. Preferred NspA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘n’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 4. The most preferred NspA antigens of the invention can elicit antibodies which, after administration to a subject, can bind to a meningococcal polypeptide consisting of amino acid sequence SEQ ID NO: 4. Advantageous NspA antigens for use with the invention can elicit bactericidal anti-meningococcal antibodies after administration to a subject.

### Nhha Antigen.

The NhhA antigen was included in the published genome sequence for meningococcal serogroup B strain MC58 (see e.g. Tettelin et al. (2000) Science 287:1809-1815) as gene NMB0992 (GenBank accession number GI:7226232; SEQ ID NO: 5 herein). The sequences of NhhA antigen from many strains have been published since e.g. WO00/66741 and WOO1/55182, and various immunogenic fragments of NhhA have been reported. It is also known as Hsf. Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 5. The most preferred NhhA antigens of the invention can elicit antibodies which, after administration to a subject, can bind to a meningococcal polypeptide consisting of amino acid sequence SEQ ID NO: 5. Advantageous NhhA antigens for use with the invention can elicit bactericidal anti-meningococcal antibodies after administration to a subject.

### App Antigen.

The App antigen was included in the published genome sequence for meningococcal serogroup B strain MC58 (see e.g. Tettelin et al. (2000) Science 287:1809-1815) as gene NMB1985 (GenBank accession number GI:7227246; SEQ ID NO: 6 herein). The sequences of App antigen from many strains have been published since then. Various immunogenic fragments of App have also been reported. Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) comprising a fragment of at least ‘n’ consecutive amino acids of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 6. The most preferred App antigens of the invention can elicit antibodies which, after administration to a subject, can bind to a meningococcal polypeptide consisting of amino acid sequence SEQ ID NO: 6.

Advantageous App antigens for use with the invention can elicit bactericidal anti-meningococcal antibodies after administration to a subject. fHbp antigen.

The factor H binding protein exists as three variants (v1, v2 and v3), and the invention can use any of these as preferred embodiment.

A v1 fHbp preferably comprises (a) an amino acid sequence which has at least k′% identity to SEQ ID NO: 8, and/or (b) a fragment of SEQ ID NO: 8. k′ refers to percentage identity and could be defined as any number from 1 to 100. With reference to amino acid or nucleic acid sequences, generally the identity used in the application starts from as low as 40% with specific references to higher percentages, i.e. 70%, 75%, 80%, etc.

The fragment will preferably include at least one epitope from SEQ ID NO: 8. Preferably, the v1 fHbp can elicit antibodies which are bactericidal against v1 strains e.g. against strain MC58 (available from the ATCC as BAA-335′).

A v2 fHbp preferably comprises (a) an amino acid sequence which has at least k′% identity to SEQ ID NO: 1, and/or (b) a fragment of SEQ ID NO: 1. Information about ‘k’ and fragments are given above. The fragment will preferably include at least one epitope from SEQ ID NO: 1.

Preferably, the v2 fHbp can elicit antibodies which are bactericidal against v2 strains e.g. against strain M2091 (ATCC 13091).

A v3 fHbp preferably comprise (a) an amino acid sequence which has at least k′% identity to SEQ ID NO: 9, and/or (b) a fragment of SEQ ID NO: 9. Information about ‘k’ and fragments are given above. The fragment will preferably include at least one epitope from SEQ ID NO: 9. Preferably, the v3 fHbp can elicit antibodies which are bactericidal against v3 strains e.g. against strain M01-240355.

Antigens from Group A Streptococcus (GAS), Group B Streptococcus (GBS) and Pneumococcus are also equally preferred. As non-limiting examples, GAS25 (Slo), GAS40 (SpyAD) and GAS57 (SpyCEP) antigens can be incorporated into conjugates in accordance with some embodiments of the invention.

Plasmodium antigens are further preferred. These can be from any suitable species, where preferred species are selected from: P.falciparum, P.vivax and P.ovale.

Still another preferred antigen is Pfs25 (SEQ ID NO: 10), which is a sexual stage antigen of P.falciparum expressed on the surface of zygote and ookinete forms of the parasite. Another preferred antigen is Pfs48/45, which is a transmission-blocking vaccine candidate. Recently the C-terminal 10 cysteine fragment (10C) of Pfs48/45, containing three known epitopes for transmission blocking antibodies, has been produced as a chimera with the N-terminal portion of GLURP (RO), the asexual blood-stage antigen glutamate-rich protein. The resulting fusion protein (RO10C) elicited high levels of transmission-blocking antibodies in rodents (see Theisen et al. (2014) Vaccine 32:2623-2630). Shing et al. (2015) Vaccine 33:1981-1986 describes a chimera containing truncated 6C-fragments, which increases the yield of correctly-folded conformer. The RO6C construct was able to elicit high titer transmission blocking antibodies in rats. RO6C (SEQ ID NO: 11) is a preferred antigen that can be conjugated according to the present invention.

Another preferred antigen is the circumsporozoite protein (CSP; SEQ ID NO: 12).

Shorter peptides from CSP may also be conjugated according to the present invention. For example, the 12 amino acid (NANP)₃ peptide (SEQ ID NO: 13) derived from CSP can be used according to preferred embodiments.

In another still preferred embodiment the antigens are a saccharide species. The invention is in fact also suitable for conjugating one or more selected saccharide antigens to nOMVs, whereby saccharides may be used in their full-length natural form. As an alternative, a particular size fraction can also advantageously be selected. Thus, a saccharide may be fragmented from their natural length, and optionally a size fraction of these fragments can be used. Even further, the saccharides are not limited to saccharides purified from natural sources and synthetic or semi-synthetic saccharides can be used instead.

Preferred saccharide antigens are bacterial capsular saccharides (CPSs). These include, but are not limited to, the capsular saccharides selected from at least one of: Haemophilus influenzae type B and type A; Neisseria meningitidis serogroups A, C, W135, X and Y; Streptococcus pneumoniae serotypes 1, 2, 3, 4, 5, 6A, 6B, 7F, 8, 9N, 9V, 10A, 11A, 12F, 14, 15B, 17F, 18C, 19A, 19F, 20, 22F, 23F, and 33F; Salmonella including Salmonella enterica serovar Typhi Vi, either full length or fragmented (indicated as fVi); Shigella sp, group A and B Streptococcus (GAS and GBS respectively).

In any case, and as above mentioned, the selected antigens could be conjugated to nOMVs derived from the same or even from a different bacterial strain, thus providing a multivalent vaccine. In this respect, in a more preferred embodiment of the invention, the nOMV and the saccharide antigen are derived from different bacterial strains.

Other preferred saccharide antigens are β-glucans, particularly useful for protecting against C.albicans (for a general reference see Sandlin et al. (1995) Infect. Immun., 63:229-37).

Other preferred saccharide antigens are poly-rhamnose oligosaccharides for protecting against Group A Streptococcus (GAS). Native GAS saccharide has a poly-rhamnose backbone substituted with NAcGIcN. Synthetic oligosaccharides of poly-rhamnose, or oligomers with the structure of native GAS saccharide, can be conjugated to nOMVs according to the invention.

As formerly set forth, in a further aspect, the invention refers to a process for preparing the present conjugates, comprising the steps of:
i) activating at least a nOMV saccharide moiety, generally bond to the nOMV surface;
ii) connecting the thus obtained activated saccharide to at least one selected antigen to obtain an antigen-nOMV intermediate;
iii) reacting at least a surface protein residue of the antigen-nMOV intermediate of the step ii) with the first terminal portion of a bivalent Linker to obtain an antigen-nOMV-Linker intermediate; and
iv) connecting said nOMV-Linker intermediate to at least one different antigen via the second terminal portion of the bivalent Linker, thus obtaining an antigen-nOMV-Linker-antigen conjugate of the invention.

According to the present process, the nOMV-surface bond saccharides are first activated by oxidation, and then reacted with the selected antigens, more preferably under reductive amination conditions. Subsequently, the thus functionalized nOMV is reacted with the linker and then connected through this latter to a different antigen.

As preferred embodiment, nOMV vesicles, preferably GMMA, from MenB are first functionalized with saccharide from MenC through linkage to saccharide moiety on the nOMV surface via reductive amination. Then, the thus obtained nOMV-MenC vesicles are conjugated to the MenA saccharide via BS3 linker targeting a protein moiety on nOMV, providing the final MenC, MenA nOMV conjugate.

As still preferred embodiment, nOMV vesicles, preferably GMMA, from MenB are first functionalized with antigen from MenA through linkage to saccharide moiety on the nOMV surface via reductive amination. Then, the thus obtained nOMV-MenA vesicles are conjugated to the MenC antigen via BS3 linker targeting a protein moiety on nOMV, providing the final MenA, MenC nOMV conjugate.

As preferred embodiment, nOMV vesicles, preferably GMMA, from S. sonnei are first functionalized with the antigen 405 through linkage to saccharide moiety on the nOMV surface via reductive amination. Then, the thus obtained nOMV-405 vesicles are conjugated to the 3526 antigen via BS3 linker targeting a protein moiety on nOMV, providing the final 405, 3526 nOMV conjugate.

As still preferred embodiment, nOMV vesicles, preferably GMMA, from S. sonnei are first functionalized with antigen from 3526 through linkage to saccharide moiety on the nOMV surface via reductive amination. Then, the thus obtained nOMV-3526 vesicles are conjugated to the 405 antigen via BS3 linker targeting a protein moiety on nOMV, providing the final 3526, 405 nOMV conjugate.

The two functionalization procedures (through saccharide and through a surface protein-linker as per steps i-ii and iii-iv respectively) can be performed in any order. This means that in a still preferred embodiment of the invention, the nOMV can be first conjugated to an antigen via a protein and linker connection, and subsequently conjugated to a different antigen via a surface saccharide moiety.

Thus, the present invention refers to a process comprising the steps of:
i) reacting at least a nMOV surface protein residue with the first terminal portion of a bivalent Linker to obtain a nOMV-Linker intermediate, and
ii) connecting said nOMV-Linker intermediate to at least one selected foreign antigen via the second terminal portion of the bivalent Linker, thus obtaining the nOMV-Linker-antigen intermediate;
iii) activating at least a nOMV saccharide moiety of the nOMV-Linker-antigen intermediate of step ii), and
iv) connecting the thus obtained activated saccharide to at least a different antigen, thus obtaining the antigen-nOMV-Linker-antigen conjugates of the invention.

According to one embodiment, the present invention refers to a process for the preparation of conjugates obtained by connecting nOMV, preferably GMMA from S. sonnei, with E. coli 405 and E. coli 356. To that, said nOMV, preferably GMMA from S. sonnei, are first connected to the E. coli 405 antigen through BS3 linker to give nOMV-BS3-E. coli 405 derivative. This latter is thus subjected to oxidation and reaction with the E. coli 3526 and NaBH₃CN, thus connecting the antigen to the nOMV vesicle through reductive amination and leading to the obtainment of the bi-functionalized nOMV conjugate in a selective and reproducible way. Of note, DLS analysis showed a size distribution of the thus obtained nOMV confirming that no aggregation occurs, as indicated in FIG. 3.

Advantageously, the process of the invention can be repeated to further conjugate the nOMV to additional different antigens, thus providing a new and reliable way to obtain multivalent immunogenic nOMV, useful for the preparation of corresponding immunogenic compositions and vaccines.

According to the present process, at least one saccharide moiety on a nOMV is conjugated to one selected antigen to form a conjugate of the invention. As above indicated, the conjugation typically involves activating the nOMV-surface saccharide moiety and/or the selected antigen.

Thus, in one embodiment, the process of the invention comprises the steps of: (i) activating a saccharide moiety on the nOMV surface by oxidation; and (ii) direct connection of the activated moiety with a selected antigen.

The conjugation can also involve introducing a linker between the nOMV-saccharide moiety and the selected antigen, as below detailed. Thus, as an alternative embodiment, the process of the invention comprises the steps of: (i) activating a saccharide moiety on the nOMV surface by oxidation; (ii) connecting said activated saccharide moiety to a bivalent linker to form a nOMV-linker intermediate; and (iii) connecting a selected antigen to said nOMV-linker intermediate, to form a nOMV-Linker-antigen conjugate.

As another alternative embodiment, the process of the invention comprises the steps of: (i) activating a saccharide moiety on the nOMV surface; (ii) connecting a selected antigen to a bivalent linker to form an antigen-linker derivative; and (iii) connecting the activated moiety of step (i) to said antigen-linker derivative to form a nOMV-Linker-antigen conjugate.

As another alternative embodiment, the process of the invention comprises the steps of: (i) activating a saccharide moiety on the nOMV surface; (ii) connecting said activated moiety to a bivalent linker to form a nOMV-linker intermediate; (iii) connecting a selected antigen to a bivalent linker group to form an antigen-linker derivative; and (iv) connecting the nOMV-linker intermediate of step (ii) to the antigen-linker derivative of step (iii) to form a nOMV-Linker-antigen conjugate of the invention.

As far as the nOMV saccharide moiety is concerned, it has to be noted that it can be part of the—OAg functionality, or of the core region naturally present on the surface of the nOMV (e.g. in LPS or LOS), or it can be present within a different nOMV surface portions, e.g. a CPS. In all these preferred cases, the process of the invention allows the connection of said saccharide moiety with a selected antigen in a simple and effective way, thus leading to final nOMV-antigen conjugates endowed with remarkable immunogenic activity. Depending on the species from which nOMVs are prepared, various saccharide moieties (including tetraose, pentose and hexose sugars) can be used for activation and subsequent conjugation. Preferably, lipopolysaccharides, via the—OAg portion or core region, or capsular saccharides may be used for activation and subsequent conjugation. Preferred saccharide moieties are selected from at least one of: glucose, galactose, fructose, mannose, ribose, abequose, galactosamine, glucosamine, mannosamine, sialic acid, sulfoquinovose, erythrose, threose, arabinose, rhamnose, sorbose, ribulose, xylose, xylulose, lyxose, tagatose or keto-deoxyoctulosonate.

A saccharide moiety on the nOMV is preferably activated by oxidizing a hydroxyl group of the saccharide to form a carbonyl aldehyde functionality, in the presence of an oxidizing agent.

Preferred oxidizing agent are TEMPO or a periodated salt. This latter is preferably selected from an alkali periodate or a metaperiodate, more preferably NalO₄. The oxidizing agent is preferably used as aqueous solution in a concentration ranging from 0.5 mM to 20 mM, preferably from 3 mM to 20 mM, where concentrations from 10 to 20 mM and from 0.5 to 5 mM or from 3 to 5 mM are still more preferred. Other activation reactions according to some embodiments occurs in the presence of: cyanylating reagents such as CDAP (e.g. 1-cyano-4-dimethylamino pyridinium tetrafluoroborate), carbodiimides, hydrazides, active esters, norborane, p-nitrobenzoic acid, N-hydroxysuccinimide, S-NHS, EDC and TSTU.

In general, where polysaccharides are oxidised it is not necessary to oxidise all of the available sugars. Indeed, it can be desirable to retain at least part of the natural sugar structures, particularly where these are a useful antigen. Also to be noted is the fact that due to the peculiar nOMVs composition and conformation as above detailed, the polysaccharide moiety can be conveniently activated by the present process leading to the formation of a highly reactive oxidized nOMVs intermediate species. In a preferred embodiment, for a given saccharide moiety of interest, the proportion of oxidised residues can range from 1% to 100%, preferably from 10-50%, or from 20-40%, or from 20-35%, whereas oxidation of 20-35% within an —OAg structure is particularly preferred. In this direction, it has been found that said ranges allow for efficient conjugation with minor or substantially assent impact on the—OAg structural integrity. Also, it has been noticed that higher nOMV oxidation degree corresponds to lower —OAg size, meaning that there is major impact on native —OAg structure and its ability to induce a specific immune response. The proportion of oxidised residues can be determined by high-performance anion exchange chromatography with pulsed amperometric detection (HPAEC-PAD), by comparing the intact sugar residues pre- and post-oxidation. In this direction, it was found that the concentration of the oxidising agent and of the nOMV, along with the pH may influence the overall conduct of the oxidation step. Thus, in a preferred embodiment, the oxidation agent is used in excess over the starting nOMV, where a molar excess of 3:1 or 2:1 respect to the number of monosaccharides that can be subjected to oxidation is particularly preferred. The oxidizing agent is preferably used as aqueous solution in a concentration ranging from 0.5 mM to 20 mM, preferably from 3 mM to 20 mM, where concentrations from 10 to 20 mM and from 0.5 to 5 or from 3 to 5 mM are still more preferred.

The concentration of nOMV is preferably comprised between 0.2 and 5 mg/mL.

Preferably, the pH is comprised between 4 and 8, whereas value from 5 and 7 are particularly preferred. To this extent, the pH may be adjusted using a buffer agent, such as acetate/phospate and the like.

Said parameters can be conveniently set in order to have a preferred degree of oxidation comprised between 20% and 35% over the subjected saccharide moiety. This allows having an efficient further conjugation with the selected antigen, without substantially impacting the saccharide moiety structure.

For instance, Rha residues in an —OAg functionality can be oxidised as e.g. indicated in the below Scheme 2 using NalO₄.

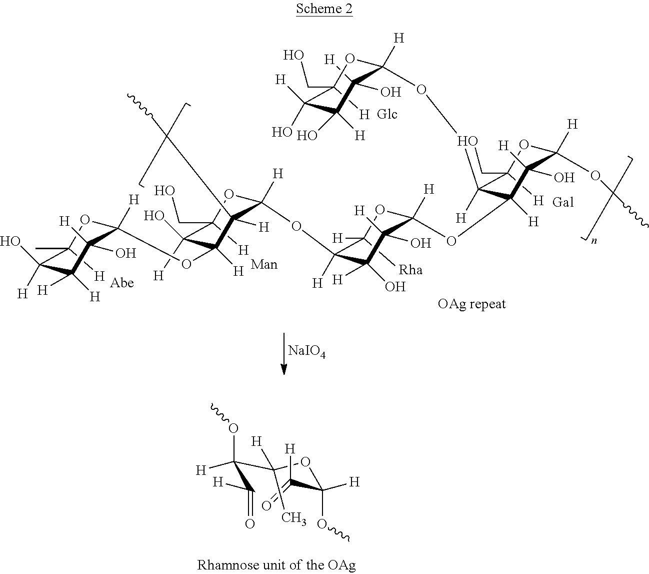

The oxidation step is typically performed at room temperature (e.g. from about 15° C. to about 40° C.), for a suitable time, e.g. comprised from 30 min to 3 h, depending for example on the amount and type of considered nOMV. In any case, it has been found that no substantial nOMV crosslink and/or aggregation occurred. This is of upmost importance also for the effectiveness of the subsequent conjugation step with the selected antigen as herein described in details.

After oxidation, nOMVs can optionally be subjected to a reduction step, for example with NaBH₄, to stabilise the oxidised nOMV by removing the formed CHO groups. The stabilised oxidised nOMV may then be stored and/or further characterised.

Typically, after the activation step of the present process, the obtained oxidized nOMVs are isolated and purified e.g. by ultracentrifugation at 4° C. at 110000 rpm for 30 min, and subsequently reacted with the selected antigen.

Thus in a preferred embodiment, the process comprises the steps of:
(i) activation of the saccharide moiety on the nOMV surface, preferably by oxidation; (i-bis) isolation of the thus obtained oxidised nOMV; and
(ii) connection of the oxidised nOMV of step (i) or (i-bis) with at least a selected antigen, optionally via a bivalent linker, to obtain a antigen-nOMV intermediate,
(iii) reacting at least a surface protein of the antigen-nOMV intermediate of step (ii) with the first terminal portion of a bivalent Linker, to obtain a antigen-nOMV-Linker intermediate, and
(iv) connecting said antigen-nOMV-Linker intermediate to at least one different antigen via the second terminal portion of the bivalent Linker, thus obtaining an antigen-nOMV-Linker-antigen conjugate of the invention.

In a still preferred embodiment, the process is performed in the presence of an alkaline sulphite, preferably Na₂SO₃. This is particularly advantageous because by quenching the oxidation reaction with Na₂SO₃, it is possible to perform the process in one step, i.e. avoiding the isolation of the intermediate oxidised nOMV (step (i-bis) above). This allows saving time, thus obtaining the final conjugates in a simple and effective way. In practice, and according to an exemplified embodiment, after the activation step (i) the reaction is quenched with a proper amount of the alkaline sulphite, and let to react for a proper frame of time (generally comprised from 5 to 20 minutes) in order to neutralize the excess of the oxidizing agent. After that, the selected antigen is directly added to the mixture (i.e. without isolation of the oxidised nOMV), according to step (ii), followed by steps (iii) and (iv) as above indicated, thus obtaining the final nOMV conjugates of the invention.

As an alternative, the carbonyl aldehyde group of the saccharide moiety obtained by the oxidation step can be further modified to form a proper functionality which can then be reacted with the selected antigen or with a linker as the case may be (in this case to give a vesicle-linker conjugate which can then be coupled to the selected antigen).

The selected antigen is typically added at 1:1 w/w ratio with respect to the used nOMV, at room temperature, for a proper frame of time, e.g. comprised from 2 hours to 24 hours. When the antigen is derivatised with a linker, the reaction is conveniently carried out using an excess of antigen over the nOMV, preferably a 2:1 or more preferably a 3:1 w/w ratio. In a particularly preferred embodiment, the process of the invention comprises the steps of: (i) oxidation of a nOMV saccharide moiety as above set forth; and (ii) connection of the thus obtained oxidized nOMV saccharide moiety to an amino group of a selected antigen residue. Even more preferably, said selected antigen residue is an amino —NH₂ group on a lysine residue within a polypeptide selected antigen.

Preferably, connection of the oxidized saccharide moiety of the nOMV with the amino group, preferably a free —NH₂ group, of an antigen is achieved by reductive amination, more preferably using NaBH₃CN, e.g. according to procedure known in the art. The NaBH₃CN is used in weight amounts (w/w) comprised from 3 to ⅓, preferably 1 to 1, over the oxidized nOMV. Practically, the NaBH₃CN can be added together with the selected antigen, directly to the oxidised nOMV intermediate product, as generally illustrated in Schemes 3 and 4 below, using, by way of example, a nOMV that is conjugated via an oxidized rhamnose unit to malaria membrane proteins

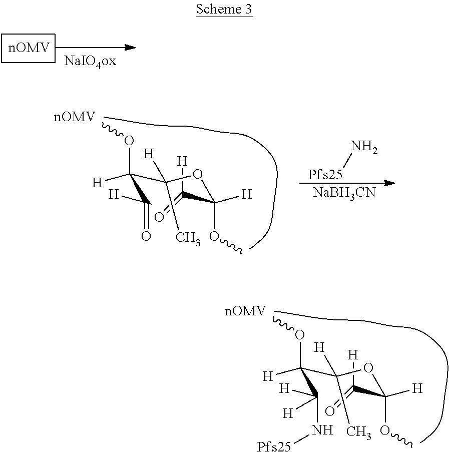

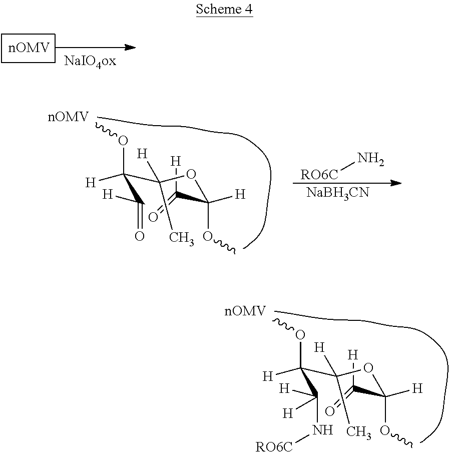

As an alternative embodiment, the selected antigen may be modified, either by introducing a linker group or by converting a functional group on the antigen into another functional group suitable for the reaction with the activated saccharide moiety on the nOMV, or with a linker of the nOMV-linker conjugate when used. In particular, if the selected antigen is a saccharide, it may be modified by reaction with a linker either randomly (r), meaning that the linker is introduced at multiple points along the sugar chain, or selectively (s), meaning that the linker is introduced at the reducing end of the sugar chain (i.e. at only one position). In a preferred embodiment, the linker is selective introduced at the terminal position of the selected antigen.

Selective modification of the antigen is preferably achieved by reaction with adipic acid dihydrazide (ADH) in the presence of NaBH₃CN, as generally shown in Scheme 5 using fVi as the antigen. Random modification of the antigen is preferably achieved by activation of one or more carboxylic acid groups on the antigen, for instance by using NHS/EDAC, and subsequent reaction with ADH, as shown in Scheme 6 using fVi as the antigen. This type of conjugation reaction is illustrated in Scheme 7 below, using, by way of example, a nOMV that is conjugated via an oxidized rhamnose unit to fVi modified to include a —NH₂ by reaction with ADH.

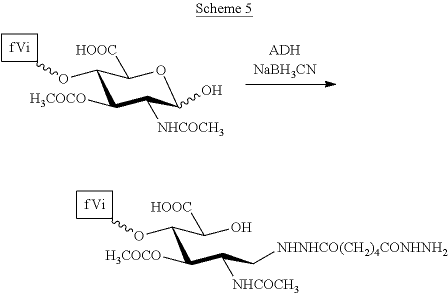

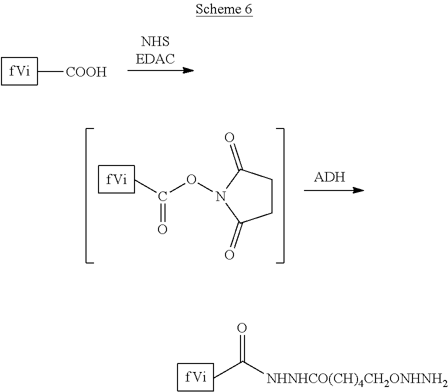

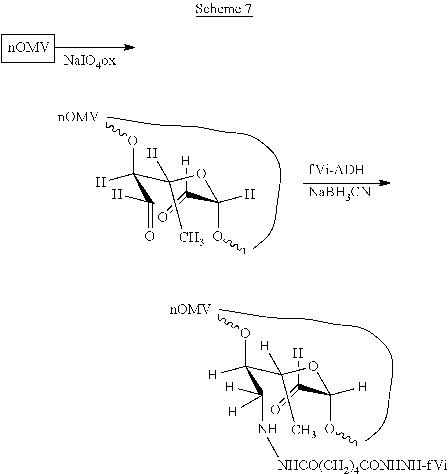

As above set forth in details, the nOMV-antigen conjugates of the invention comprise an activated nOMV surface saccharide moiety directly connected to a selected antigen.

In an equally preferred embodiment, the activated nOMV surface saccharide moiety is connected to the selected antigen indirectly, e.g. via a linker unit. This latter will generally be a bifunctional linker, using one functional group to react with the nOMV (via the activated saccharide moiety) and another functional group to react with the selected antigen. The linker can be a heterobifunctional linker or a homobifunctional linker of general formula (I):

wherein:

X and X′ groups are independently the same or different as each other, and react with activated nOMV surface saccharide moiety and the selected antigen respectively; and L is a linking spacer, preferably of general formula (II):

-L′-L2-L′- (II)

wherein:

the two L′ groups are independently the same or different as each other and are selected from: a carbonyl (C═O), ester (—C(O)O—) or amido group (—C(O)NR1—), wherein R1 is H or, a straight, or, when comprising at least 3 carbon atoms, a branched cyclic C1-C10 alkyl group having 1 to 10 carbon atoms (e.g. C₁, C2, C₃, C4, C5, C6, C7, C8, C9, C10); and L2 is a straight or branched C₁-C10 alkyl group having 1 to 10 carbon atoms, preferably having C4 carbon atom, even more preferably in the form of a straight chain.

X group is preferably selected from: —NH₂, —NH—NH₂, —O—NH₂, optionally substituted sulfo-N-hydroxysuccinimide and N oxysuccinimide residue.

Where the reactions with both the nOMV and the selected antigen involve the same functional groups it is preferred to use a bifunctional linker of general formula (I), wherein both the two X groups are the same.

When the functional groups on the nOMV saccharide moiety and on the selected antigen are both aldehydes it is preferred to use a homofunctional linker having X selected from: —NH₂, —NH—NH₂ or —O—NH₂ reactive group. In a still preferred embodiment, the linker is the adipic acid dihydrazide (ADH) of general formula:

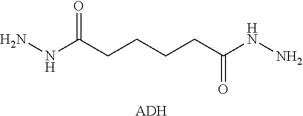

The linker may then be reacted with the nOMV and/or antigen by reductive amination as above set forth.

Preferred bifunctional linkers particularly useful for the reaction with amine groups of the selected antigen, are selected from: acryloyl halides, preferably chloride, disuccinimidyl glutarate, disuccinimidyl suberate and ethylene glycol bis[succinimidylsuccinate].

Other still preferred linkers are selected from: β-propionamido, nitrophenyl-ethylamine, haloacyl halides, glycosidic derivatives linkages, 6-aminocaproic acid.

In a still preferred embodiment, the linker is selected from: N-hydroxysuccinimide, N oxysuccinimide, even more preferably from adipic acid N-hydroxysuccinimide diester (SIDEA).

When the reaction with the nOMV and the antigen involves different functional groups (such as an amine on the nOMV and a thiol on the antigen,) it will be understood that a heterobifunctional linker will be used capable to selectively react with both the different functional groups. In this case, preferred heterobifunctional linkers are selected from at least one of: succinimidyl 3-(2-pyridyldithio)propionate (SPDP), succinimidyl 6-(3-[2-pyridyldithio]-propionamido)hexanoate (LC-SPDP), sulfosuccinimidyl 6-(3′-(2-pyridyldithio)propionamido)hexanoate (sulfo-LC-SPDP), 4-succinimidyloxycarbonyl-α-methyl-α-(2-pyridyldithio)toluene (SMPT), sulfosuccinimidyl-6-[α-methyl-α-(2-pyridyldithio)tolueamideo]hexanoate (sulfo-LC-SMPT), succinimidyl 4-(N-maleimidomethyl)cyclohexane-1-carboxylate (SMCC), sulfosuccinimidyl 4-(N-maleimidomethyl)cyclohexane-1-carboxylate (suflo-SMCC), m-maleimidobenzoyl-N-hydroxysuccinimide ester (MBS), m-maleimidobenzoyl-N-hydroxysulfosuccinimide ester (sulfo-MBS), N-succinimidyl (4-iodoacetyl)aminobenzoate (SIAB), sulfosuccinimidyl (4-iodoacetyl)aminobenzoate (sulfo-SIAB), succinimidyl 4-(N-maleimidophenyl)butyrate (SMPB), sulfosuccinimidyl 4-(N-maleimidophenyl)butyrate(sulfo-SMPB), N-γ-maleimidobutyryl-oxysuccinimide ester (GMBS), N-γ-maleimidobutyryl-oxysulfosuccinimide ester (sulfo-GMBS), succinimidyl-6-((((4-(iodoacetyl)amino)methyl)cyclohexane-1-carbonyl)amino)hexanoate (SIACX), succinimidyl 6[6-(((iodoacetyl)amino)hexanoyl)amino]hexanoate (SIAXX), succinimidyl-4-(((iodoacetyl)amino)methyl)cyclohexane-1-carboxylate (SIAC), and succinimidyl 6-[(iodoacetyl)amino]hexanoate (SIAX) and β-nitrophenyl iodoacetate (NPIA).

In a further embodiment, the process encompasses the possibility to recycle the unreacted selected antigen particularly when in form of polypeptide. To this extent, it has been found that the unreacted antigen from the conjugation mixture can be conveniently recycled in the conjugation step, thus improving the overall efficiency of production of the derivatives of the invention.

As above set forth, the invention refers to a process for preparing multi-functionalized nOMV-antigen conjugates, comprising the reaction of at least a nOMV protein residue with a first terminal portion of a bivalent Linker, according to the above indicated formula (I) X-L-X′, followed by reaction of the second terminal portion of such bivalent Linker with one or more selected foreign antigens, as herein below described in more details. Of note, this selective functionalization can be performed before or after the functionalization of the same nOMV with a different antigen via a polysaccharide residue, according to the above described embodiment.

Where the reactions with the protein on the surface of the vesicle and the antigen involve different functional groups (such as an amine on the protein on the vesicle and a thiol on the antigen, or vice versa) it is preferred to use a heterobifunctional Linker of the above general formula (I) X-L-X′, where X and X′ are different to each other and as above defined and L is a moiety as above defined. The X group can react with one functional group, e.g. an amine on the nOMV protein; whereas X′ group can react with a different functional group, e.g. a thiol on the selected antigen.

Preferably, the X group is N-hydroxysuccinimide or N-oxysuccinimide or derivatives thereof, whereas the X′ group is selected from at least one of: 2-pyridyldithio, maleimide or iodoacetyl group.

The percentage of nOMV-Linker functionalization is comprised from 15% to 60%, mainly depending on the kind of bivalent linker used. To this regard, the % of reactive functional groups at the free terminal end of the Liker is comprised from 15% to 40%, preferably from 30% to 35%, depending on the kind and stability of said reactive functional groups. It is in fact noticed that such ranges allow for an implemented efficacy of the process, thus resulting in higher amount of final antigen conjugate derivative of the invention.

The thus obtained buffered suspension has a nOMV concentration comprised from 2 and 10 mg/mL, preferably from 3 to 6 mg/mL. The chosen Linker, is generally added in amounts depending e.g. on the—NH₂ groups on the nOMV, preferably in excess, even more preferably comprised from 10 to 20 equivalents per mole of —NH₂.

Depending on the Linker, it could be convenient to preventively solve it in a polar dry solvent, such as DMSO or the like, in order to facilitate the handling and the efficacy, thus obtaining improved results in terms of overall yield and reproducibility.

The mixture is then incubated at room temperature (e.g. comprised from about 15 to 40° C.) for a period of time generally comprised from 30 minutes to 4 hours. Subsequently, the thus obtained nOMV-Linker intermediate is purified, e.g. by ultracentrifugation, and then reacted with the selected antigen according to step ii). The antigen is generally solved in a proper buffer solution, such as phosphate buffered saline. The antigen is preferably added in an amount ranging from 2:1 to 1:2 w/w ratio over the intermediated activated nOMV or more preferably in a 1:1 ratio. The reaction is carried out for a proper frame of time, up to the formation of the final nOMV-Linker-antigen derivative according to the invention. The reaction can be monitored e.g. by HPLC/SEC and the final product formation can be confirmed by SDS page/western blot analysis.

The skilled person will understand that when a homobifunctional Linker is used, the protein and the antigen functional groups involved in the reaction will be the same chemical entity, as above explained in details.

Thus, according to one preferred embodiment, the process of the present invention comprises the steps of:
(i) reaction of a —NH₂ group of at least a nOMV protein surface with a bivalent homobifunctional Linker to form a nOMV-Linker intermediate, wherein the nOMV has been previously functionalized with a selected antigen, through a saccharide moiety as above set forth; and
(ii) connection of said intermediate with a —NH₂ group of one or more selected different antigen(s) to form a conjugate of the invention.

An alternative conjugation process of the invention includes reacting the antigen with a first Linker and a protein on the vesicle with a second Linker, then reacting the first and second Linker together to form the conjugate.

By way of example, either the antigen or the protein on the OMV may be reacted with a Linker terminating in a maleimide group, for instance by reacting a primary amine or either the antigen or the protein on the nOMV with succinimidyl-4-(N-maleimidomethyl)cyclohexane-1-carboxylate (SMCC) or N-(γ-maleimidobutyryloxy)succinimide ester (GMBS). A thiol on either the antigen or the protein on the nOMV may then be reacted with the maleimide. The thiol may be native to the protein on the nOMV or antigen or the thiol may be the result of reacting the protein on the nOMV or antigen with a separate Linker. This type of conjugation reaction is exemplified in Scheme 8 below, using, by way of example, GMMA and fHbp as the antigen:

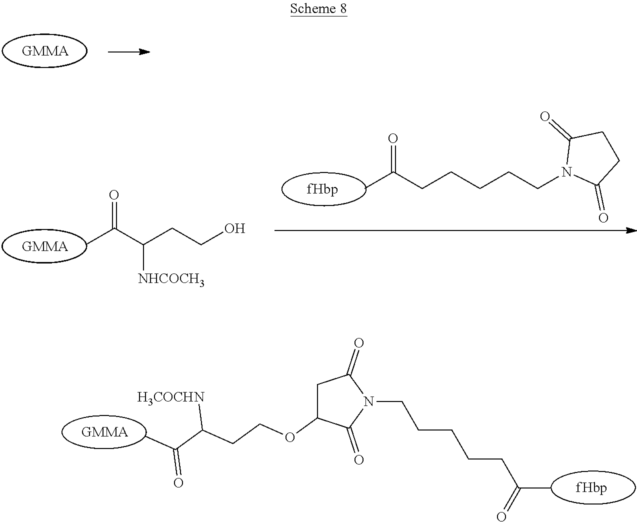

Advantageously, due to its versatility, the invention can be used for the preparation of a variety of conjugates, particularly appreciated by the skilled person when faced with the problem to find convenient and reliable methodologies for obtaining immunogenic derivatives.

As a further alternative, a protein on the nOMV may be linked to the antigen by (i) modifying the nOMV protein to include an alkyne; (ii) modifying the antigen to include an azide, then (iii) reacting the alkyne and azide, known as “click chemistry”. Alternatively, the antigen may be modified to include an alkyne and the vesicle may be modified to include an azide. This type of conjugation reaction is illustrated in Scheme 9 below, using, by way of example, GMMA as the vesicle and fHbp as the antigen and using copper free click chemistry.

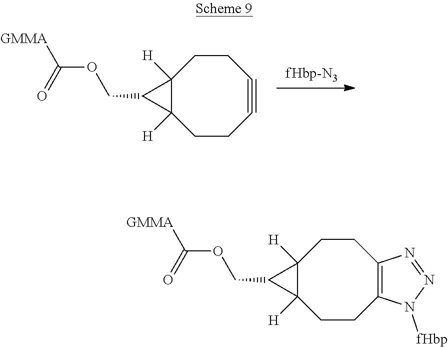

According to a further aspect, the invention refers to the above described nOMV conjugates for use as medicament, particularly as immunogenic agents, even more preferably for one or more of the pathogens as herein indicated. In other words, the invention refers to the use of the present nOMV conjugates for the manufacture of an immunogenic composition, preferably a vaccine.

As above explained in details, the present process allows the preparation of the desired immunogenic nOMV conjugates in a simple and convenient way, also requiring fewer steps when compared to previous methods for the preparation of similar conjugates(e.g. starting from dOMV).

Thus the invention also refers to a nOMV conjugates obtained (or obtainable) by the process of the invention, according to the above described embodiments. Particularly, the present process does not necessarily require the expensive step of polypeptide antigen derivatisation, as well as not performing an extraction (e.g. using a detergent) or denaturation of the starting vesicles.

Production and purification of nOMVs of the invention in fact is less expensive than for traditional carrier proteins and more robust and consistent than production of dOMV. nOMV used in the invention can be produced at high yields using e.g. two simple tangential flow filtration steps, and avoiding detergent extraction procedures. Also, the present invention offers an easy way to prepare a polyvalent immunogenic composition, e.g. a vaccine which includes multiple immunogens (typically from different pathogens) by properly choosing the nOMV and the selected antigens as herein described in more details. In fact, due to its versatility, the present process may be conveniently and effectively applied to nOMV from different sources (e.g. Salmonella, Shigella and meningococcal), being applied with success to both protein and saccharide antigens, according to the presently described selective conjugations. Finally, it has to be noted that the present process not only allows for the preparation of highly immunogenic derivatives, but also it does not substantially change the nOMV integrity and size distribution. This is particularly appreciated by the skilled in the art, because the absence of nOMV aggregates allows for a better yield and overall consistency and robustness of the present process.

Conjugates of the invention which include nOMVs from one pathogen and at least two different antigens from a second pathogen can be useful as immunogenic composition, preferably as multivalent vaccines. Pairs of pathogens which may be combined (one as antigen, and the other as a nOMV vesicle) include, but are not limited to: N. meningitidis and non-typhoidal Salmonella (e.g. Salmonella Typhimurium or Salmonella Enteritidis); P. falciparum and non-typhoidal Salmonella; Salmonella Typhi and non-typhoidal Salmonella; E. coli and Shigella sp.; Group A Streptococcus (GAS) and N. meningitidis; and GAS and non-typhoidal Salmonella, Hib and N. meningitidis, Hib and Pertussis.

Preferred nOMV-polysaccharide-antigen combinations of the invention are indicated in the following Table 1.

TABLE 1
preferred nOMV-Antigen conjugates obtained by
oxidation of nOMV polysaccharide residues

| nOMV | Antigen |
|---|---|
| Salmonella Typhimurium | Neisseria meningitidis fHbp |
| Salmonella Typhimurium | Plasmodium falciparum CSP |
| Salmonella Typhimurium | Plasmodium falciparum Pfs25 |
| Salmonella Typhimurium | Plasmodium falciparum RO6C |
| Salmonella Typhimurium | Plasmodium falciparum RO10C |
| Salmonella Typhimurium | Escherichia coli CTF1232 |
| Salmonella Typhimurium | S. Typhi Vi saccharide |
| Neisseria meningitidis | Neisseria meningitidis fHbp |
| Neisseria meningitidis | Neisseria meningitidis NHBA |
| Neisseria meningitidis | Poly-rhamnose oligosaccharide |
| Shigella | Escherichia coli CTF1232 |
| Shigella | Escherichia coli FdeC |
| Shigella | Escherichia coli SsIE |
| Salmonella Typhimurium | Synthetic or native GAS PS |
| Neisseria meningitidis | Synthetic or native GAS PS |
| Salmonella Typhimurium | Synthetic or native GBS PS |
| Neisseria meningitidis, | Neisseria meningitidis ser A saccharide |
| preferably B | |
| Neisseria meningitidis, | Neisseria meningitidis ser C saccharide |
| preferably B | |
| Salmonella Typhimurium | Neisseria meningitidis ser A saccharide |
| Salmonella Typhimurium | Neisseria meningitidis ser C saccharide |
| B. pertussis | Haemophilus influenzae type b |
| Neisseria meningitidis, | Haemophilus influenzae type b |
| preferably B | |
| B. pertussis | Haemophilus influenzae type a |
| Neisseria meningitidis, | Haemophilus influenzae type a |
| preferably B | |
| Salmonella Typhimurium | Streptococcus pneumoniae saccharide |
| Neisseria meningitidis | Streptococcus pneumoniae saccharide |

Preferred nOMV-Linker-antigen conjugates of the invention are indicated in the following Table 2.

TABLE 2
preferred nOMV-Linker-Antigen conjugates obtained by connection
of a nOMV surface to a selected antigen via a Linker

| nOW | Linker | Antigen |
|---|---|---|
| Salmonella Typhimurium | BS3 | Plasmodium falciparum Pfs25 |
| Salmonella Typhimurium | BS(PEG)₅ | Plasmodium falciparum Pfs25 |
| Salmonella Typhimurium | BS3 | fHbp (Neisseria meningitidis) |
| Salmonella Typhimurium | BS(PEG)₅ | fHbp (Neisseria meningitidis) |
| Salmonella Typhimurium | BS3 | Plasmodium falciparum |
| | | RO6C |
| Salmonella Typhimurium | BS(PEG)₅ | Plasmodium falciparum |
| | | RO6C |
| Salmonella Typhimurium | BS3 | Plasmodium falciparum CSP |
| Salmonella Typhimurium | BS(PEG)₅ | Plasmodium falciparum CSP |
| Meningococcal B | BS3 | fHbp (Neisseria meningitidis) |
| Meningococcal B | BS(PEG)₅ | fHbp (Neisseria meningitidis) |
| Meningococcal B | BS3 | NHBA (Neisseria meningitidis) |
| Meningococcal B | BS(PEG)₅ | NHBA (Neisseria meningitidis) |
| Salmonella Typhimurium | BS3 | Synthetic or native GAS PS |
| Salmonella Typhimurium | BS(PEG)₅ | Synthetic or native GAS PS |
| Salmonella Typhimurium | BS3 | Synthetic or native GBS PS |
| Meningococcal B | BS3 | Synthetic or native GAS PS |
| Meningococcal B | BS3 | Hib PS |
| B. pertussis | BS3 | Hib PS |
| Meningococcal B | BS3 | Hia PS |
| B. pertussis | BS3 | Hia PS |
| Salmonella Typhimurium | BS3 | Vi PS |
| Shigella | BS3 | E. coli FdeC |
| Shigella | BS3 | E. coli SsIE |
| Shigella | BS3 | E. coli CTF1232 |
| Meningococcal B | BS3 | Neisseria meningitidis ser C |
| | or SIDEA | saccharide |
| Meningococcal B | BS3 | Neisseria meningitidis ser A |
| | or SIDEA | saccharide |
| Salmonella Typhimurium | BS3 | Neisseria meningitidis ser C |
| | or SIDEA | saccharide |
| Salmonella Typhimurium | BS3 | Neisseria meningitidis ser A |
| | or SIDEA | saccharide |
| Salmonella Typhimurium | BS3 | Streptococcus pneumoniae |
| | or SIDEA | saccharide |
| Neisseria meningitidis | BS3 | Streptococcus pneumoniae |
| | or SIDEA | saccharide |

Thus, the present nOMV-antigen derivatives are particularly useful as immunogenic agents against the pathogens listed in Table 1 and 2.

Of note, any combination of the antigen listed in Table 1 and in Table 2 is encompassed by the resent invention as preferred embodiments.

Still further preferred multi-functionalization patterns of the nOMV according to the present invention are indicated in Table 3:

| nOMV | Antigen 1 (via PS) | Antigen2/linker |
|---|---|---|
| Salmonella Typhimurium | Pfs25 | (NANP)₃ |
| Meningococcal B | MenA | MenC/SIDEA or BS3 |
| Meningococcal B | MenC | MenA/SIDEA or BS3 |
| Salmonella Typhimurium | MenA | MenC/SIDEA or BS3 |
| Salmonella Typhimurium | MenC | MenA/SIDEA or BS3 |
| Salmonella Typhimurium | Native or synthetic GAS PS | Neisseria meningitidis |
| | | fHbp/BS3 |
| Salmonella Typhimurium | Neisseria meningitidis fHbp | Native or synthetic GAS |
| | | PS/BS3 |
| Meningococcal B | Hia PS | Hib PS/SIDEA or BS3 |
| B. Pertussis | Hia PS | Hib PS/SIDEA or BS3 |
| Salmonella Typhimurium | Hia PS | Hib PS/SIDEA or BS3 |
| Meningococcal B | Hib PS | Hia PS/SIDEA or BS3 |
| B. Pertussis | Hib PS | Hia PS/SIDEA or BS3 |
| Salmonella Typhimurium | Hib PS | Hia PS/SIDEA or BS3 |
| Shigella | SsIE | FdeC/BS3 |
| Shigella | FdeC | SsIE/BS3 |

Thus, the present nOMV-antigen derivatives are particularly useful as immunogenic agents against the pathogens listed in Tables 1, 2 and 3.

In table 1, 2 and 3, the listed nOMVs are preferably GMMA.

In a further embodiment the invention provides a composition comprising a conjugate of the invention and at least two antigens selected from the following:
a saccharide antigen from N. meningitidis serogroup A, C, W135 and/or Y,
a saccharide antigen from Streptococcus pneumonia,
an antigen from hepatitis A virus, such as inactivated virus,
an antigen from hepatitis B virus, such as the surface and/or core antigens,
a diphtheria antigen, such as a diphtheria toxoid e.g. the CRM197 mutant,
a tetanus antigen, such as a tetanus toxoid,
an antigen from Bordetella pertussis, such as pertussis holotoxin (PT) and filamentous haemagglutinin (FHA) from B.pertussis, optionally also in combination with pertactin and/or agglutinogens 2 and 3,
a saccharide antigen from Haemophilus influenzae A or B,
polio antigen(s) such as IPV,
measles, mumps and/or rubella antigens,
influenza antigen(s), such as the haemagglutinin and/or neuraminidase surface proteins,
an antigen from Moraxella catarrhalis,
an protein antigen from Streptococcus agalactiae (group B streptococcus),
a saccharide antigen from Streptococcus agalactiae (group B streptococcus),
an antigen from Streptococcus pyogenes (group A streptococcus),
an antigen from Staphylococcus aureus.

Advantageously in this respect, the thus functionalized GMMA vesicle can be used for the preparation of trivalent vaccine against Meningitidis Serogroups B, C and A. Since the different nature of the three serogroups, the present combination is particularly valuable since it can provide a valid multivalent immunogenic composition, usable against these different kinds of pathogens.

According to a further aspect, the invention refers to the above described nOMV-antigen conjugates for use as a medicament, particularly as immunogenic agent, even more preferably for one or more of the pathogens as herein indicated. In other words, the invention refers to the use of the present nOMV-antigen conjugated derivative for the manufacture of an immunogenic composition.

According to a further aspect, the invention thus refers to an immunogenic composition, preferably a vaccine, comprising a conjugate of the invention and at least one additional pharmaceutically acceptable carrier, excipient or adjuvant. Generally, pharmaceutically acceptable carrier or excipient, can be any substance that does not itself induce the production of antibodies harmful to the patient receiving the composition, and which can be administered without undue toxicity.

Pharmaceutically acceptable carriers and excipient are those used in the art, and can include liquids such as water, saline, glycerol and ethanol. Auxiliary substances, such as wetting or emulsifying agents, pH buffering substances, and the like, can also be present in such vehicles, according to the prior art.

The invention also provides a method for raising an immune response in a vertebrate, preferably a mammal, comprising administering a conjugate of the invention to the mammal or other vertebrate. The invention also provides conjugates of the invention for use in such methods. The immune response is preferably protective and preferably involves antibodies. The method may raise a booster response.

The mammal is preferably a human. The subject in which disease is prevented may not be the same as the subject that receives the conjugate of the invention. For instance, a conjugate may be administered to a female (before or during pregnancy) in order to protect offspring (so-called ‘maternal immunisation’). Conjugates of the invention may also be used to immunise other mammals e.g. cattle, sheep and pigs (especially against Salmonella sp.), and other non-mammal vertebrates including fish and poultry.

The invention provides conjugates of the invention for use in therapy (e.g. as immunogenic compositions or as vaccines). The invention also provides a conjugate of the invention for use in a method for raising an immune response in a vertebrate, preferably a mammal. The invention also provides the use of a conjugate of the invention in the manufacture of a medicament for raising an immune response in a vertebrate, preferably a mammal. The uses and methods are particularly useful for preventing/treating a variety of diseases, depending on the antigens and nOMVs within the conjugates as above set forth. Preferred conjugates of the invention can confer an antibody titre in a patient that is superior to the criterion for seroprotection for each antigenic component for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Immunogenic compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration is preferred e.g. to the thigh or the upper arm. Injection may be via a needle (e.g. a hypodermic needle), but needle-free injection may alternatively be used. A typical intramuscular dose is about 0.5 ml. The invention may also be used to elicit systemic and/or mucosal immunity. Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

Infections affect various areas of the body and so the compositions of the invention may be prepared in various forms. For example, the compositions may be prepared as injectable, either as liquid solutions or suspensions. Solid forms suitable for solution in, or suspension in, liquid vehicles prior to injection can also be prepared. The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared for nasal, aural or ocular administration e.g. as drops. Compositions suitable for parenteral injection are most preferred. The composition is preferably sterile. It is preferably pyrogen-free. It is preferably buffered e.g. at between pH 6 and pH 8, generally around pH 7. Compositions of the invention may be isotonic with respect to humans. Immunogenic compositions comprise an immunologically effective amount of a conjugate of the invention, as well as any other of other specified components, as needed. Dosage treatment may be a single dose schedule or a multiple dose schedule (e.g. including booster doses). The composition may be administered in conjunction with other immunoregulatory agents.

Adjuvants which may be used in compositions of the invention include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolones, or mixtures thereof. Other substances that act as immunostimulating agents are disclosed for instance in Watson, Pediatr. Infect. Dis. J. (2000) 19:331-332. The use of an aluminium hydroxide and/or aluminium phosphate adjuvant is particularly preferred. These salts include oxyhydroxides and hydroxyphosphates. The salts can take any suitable form (e.g. gel, crystalline, amorphous, etc.).

The invention will be now described by the following experiment part, without posing any limitation to its scope.

### Experimental Part

### EXAMPLE 1: nOMV Production

Preferred nOMV vesicles were prepared from AtoIR strains of S. Typhimurium or S. sonnei, as e.g. disclosed in Clin Vaccine Immunol. 2016 April; 23(4): 304-314 and PLoS One. 2015; 10(8): e0134478 respectively.

Characteristics of the purified nOMV vesicles were as indicated in the following Table 1:

TABLE 1
characteristics of nOMV vesicles prepared from ΔtolR
strains of S. Typhimurium or S. sonnei.

| | S. Typhimurium 1418 | S. sonnei 1790 |
|---|---|---|
| | (ΔtolhR) | (ΔtolR ΔhtrB) |
| Diameter (nm) | 131.5 | 140 |
| Surface charge (mV) | −14.1 | −9.87 |
| Lipid A/mg vesicles | 172.8 | 155.4 |
| OAg/total protein weight ratio | 0.84 | 0.039 |

### EXAMPLE 2: Functionalization of GMMA from S. sonnei with E. coli 405 (FdeC) Via BS3 Chemistry and with E. coli 3526 (SsIE) Via Reductive Amination

GMMA from S. sonnei have been activated with BS3 linker, under the following conditions:
pH: 9 (100 mM borate buffer);
BS3 concentration: 50 mg/mL;
GMMA concentration: 4 mg/mL;
30 min reaction time at 25° C.;
Purification by UC (110000 rpm, 16 min, 4° C.).

Resulting GMMA had 43.8% of NH₂ groups derivatised with the BS3 linker according to TNBS colorimetric method. Purified activated GMMA were added of 405 protein antigen in PBS buffer with a w/w ratio of GMMA to 405 of 1:1 and a GMMA concentration of 6.45 mg/mL. After overnight gently mixing at room temperature, the conjugate (S. sonnei GMMA BS3-405) was purified by ultracentrifuge (110000 rpm, 4° C. 1h) and resuspended in NaPi 100 mM pH 6.5 for further conjugation step. The conjugate was characterised by SDS PAGE/western blot, confirming conjugate formation (FIG. 2). S. sonnei GMMA BS3-405 conjugate at the concentration of 2.1 mg/mL was incubated with NalO₄ 5 mM for 30 minutes at 25° C., in the dark. Excess of NalO₄ was quenched with Na₂SO₃ at a final concentration of 10 mM, for 15 minutes at room temperature. 3526 protein antigen (w/w ratio of GMMA conjugate to 3526 1:1 and with GMMA concentration of 1.18 mg/mL) and NaBH₃CN (few mg) were directly added to the reaction mixture. After overnight gently mixing at room temperature, the conjugate (S. sonnei GMMA BS3-405)ox-3526) was purified by ultracentrifuge (110000 rpm 4° C. 1h), re-suspended in PBS and analysed by SDS PAGE/western blot, that confirmed both presence of 405 and 3526 antigens on GMMA particles (FIG. 2).

### EXAMPLE 3a-c: Comparative Examples

### Example 3a: Reaction of dOMV (from Neisseria meningitidis B) with fHbp v3 (No Reaction)

The dOMVs of the present example have been prepared by a detergent extraction process, where the deoxhycholate is used as selected detergent. The thus obtained detergent extracted vesicles have been reacted with the selected antigen (fHbp) according to process of the present invention.

In particular, dOMV, at the concentration of 0.96 mg/mL, were incubated with NalO₄ 10 mM for 30 minutes at room temperature, in the dark. Excess of NalO₄ was quenched with Na₂SO₃ at a final concentration of 20 mM, for 15 minutes at room temperature. fHbp (w/w ratio of dOMV to fHbp 1:1 and with dOMV concentration of 0.335 mg/mL) and NaBH₃CN (3 mg) were directly added to the reaction mixture. After overnight gently mixing at room temperature, the crude was purified by ultracentrifuge (110000 rpm 4° C. 1h) and re-suspended in PBS. Analysis by SDS PAGE/western blot showed no dOMV-fHbp conjugate formation.

### Example 3b: Reaction of nOMV (from Neisseria meningitidis B) with fHbp v3 (Formation of the nOMV-fHbp Intermediate)

The nOMVs of the present example have been prepared without using any detergent, as described in Koeberling et al. Vaccine (2014) 32:2688. The thus obtained extracted vesicles have been reacted with the selected antigen (fHbp) according to the process of the present invention.

In particular, nOMV at the concentration of 0.96 mg/mL were incubated with NalO₄ 5 mM for 30 minutes at room temperature, in the dark. Excess of NalO₄ was quenched with Na₂SO₃ at a final concentration of 20 mM, for 15 minutes at room temperature. fHbp (w/w ratio of dOMV to fHbp 1:1 and with dOMV concentration of 0.335 mg/mL) and NaBH₃CN (3 mg) were directly added to the reaction mixture. After overnight gently mixing at room temperature, the conjugate was purified by ultracentrifuge (110000 rpm 4° C. 1h), re-suspended in PBS and analysed by SDS PAGE/western blot, confirming the formation of the desired nOMV-fHbp conjugate.

### Example 3c: Reaction of nOMV (from Salmonella) with fHbp v1, Following the Procedure of Example 3b

The same experiment as Examples 3b has been performed using nOMV from Salmonella Typhimurium, and similar results have been collected, obtaining the nOMV-fHbp conjugate of the invention.

### Example 4 (Comparative): dOMV Conjugation Via BS3 Linker

dOMV (from MenB) has been tested as starting material for reaction with BS3 linker, under the following conditions:
pH: 6.5;
BS3 concentration: 50 mg/mL;
dOMV concentration: 1.011 mg/mL;
30 min reaction time at 25° C.;
Purification by UC (110 Krpm, 16 min, 4° C.).

The reaction provides dOMV aggregates and side products as major results.

Aggregation/crosslinking has been verified by dls analysis and SEC/MALS.

The comparative example shows that independently from the order by which the process for multiple functionalization is intended (i.e. first connecting the vesicle to an antigen via saccharide residue, followed by subsequent connection to a second antigen via protein, or vice versa), when dOMVs are considered as vesicles of choice, no functionalization is possible since even at very early stages, the reaction only provides aggregate derivatives as main product.

### SEQUENCE LISTING

SEQUENCE LISTING

>SEQ ID NO: 1 [fHbp v2]
VAADIGAGLADALTAPLDHKDKSLQSLTLDQSVRKNEKLKLAAQGAEKTYGNGDSLNTGKLKN
DKVSRFDFIRQIEVDGQLITLESGEFQIYKQDHSAVVALQIEKINNPDKIDSLINQRSFRVSGLGG
EHTAFNQLPDGKAEYHGKAFSSDDAGGKLTYTIDFAAKQGHGKIEHLKTPEQNVELAAAELKA
DEKSHAVILGDTRYGSEEKGTYHLALFGDRAQEIAGSATVKIGEKVHEIGIAGKQ
>SEQ ID NO: 2 [NHBA]
MFKRSVIAMACIFALSACGGGGGGSPDVKSADTLSKPAAPVVSEKETEAKEDAPQAGSQGQG
APSAQGSQDMAAVSEENTGNGGAVTADNPKNEDEVAQNDMPQNAAGTDSSTPNHTPDPNM
LAGNMENQATDAGESSQPANQPDMANAADGMQGDDPSAGGQNAGNTAAQGANQAGNNQA
AGSSDPIPASNPAPANGGSNFGRVDLANGVLIDGPSQNITLTHCKGDSCSGNNFLDEEVQLKS
EFEKLSDADKISNYKKDGKNDKFVGLVADSVQMKGINQYIIFYKPKPTSFARFRRSARSRRSLP
AEMPLIPVNQADTLIVDGEAVSLTGHSGNIFAPEGNYRYLTYGAEKLPGGSYALRVQGEPAKGE
MLAGAAVYNGEVLHFHTENGRPYPTRGRFAAKVDFGSKSVDGIIDSGDDLHMGTQKFKAAIDG
NGFKGTWTENGSGDVSGKFYGPAGEEVAGKYSYRPTDAEKGGFGVFAGKKEQD
>SEQ ID NO: 3 [NadA]
MKHFPSKVLTTAILATFCSGALAATSDDDVKKAATVAIVAAYNNGQEINGFKAGETIYDIGEDGTI
TQKDATAADVEADDFKGLGLKKVVTNLTKTVNENKQNVDAKVKAAESEIEKLTTKLADTDAALA
DTDAALDETTNALNKLGENITTFAEETKTNIVKIDEKLEAVADTVDKHAEAFNDIADSLDETNTKA
DEAVKTANEAKQTAEETKQNVDAKVKAAETAAGKAEAAAGTANTAADKAEAVAAKVTDIKADIA
TNKADIAKNSARIDSLDKNVANLRKETRQGLAEQAALSGLFQPYNVGRFNVTAAVGGYKSESA
VAIGTGFRFTENFAAKAGVAVGTSSGSSAAYHVGVNYEW
>SEQ ID NO: 4 [NspA]
MKKALATLIALALPAAALAEGASGFYVQADAAHAKASSSLGSAKGFSPRISAGYRINDLRFAVDY
TRYKNYKAPSTDFKLYSIGASAIYDFDTQSPVKPYLGARLSLNRASVDLGGSDSFSQTSIGLGVL
TGVSYAVTPNVDLDAGYRYNYIGKVNTVKNVRSGELSAGVRVKF
>SEQ ID NO: 5 [NhhA]
MNKIYRIIWNSALNAWVVVSELTRNHTKRASATVKTAVLATLLFATVQASANNEEQEEDLYLDP
VQRTVAVLIVNSDKEGTGEKEKVEENSDWAVYFNEKGVLTAREITLKAGDNLKIKQNGTNFTYS
LKKDLTDLTSVGTEKLSFSANGNKVNITSDTKGLNFAKETAGTNGDTTVHLNGIGSTLTDTLLNT
GATTNVTNDNVTDDEKKRAASVKDVLNAGWNIKGVKPGTTASDNVDFVRTYDTVEFLSADTKT
TTVNVESKDNGKKTEVKIGAKTSVIKEKDGKLVTGKDKGENGSSTDEGEGLVTAKEVIDAVNKA
GWRMKTTTANGQTGQADKFETVTSGTNVTFASGKGTTATVSKDDQGNITVMYDVNVGDALNV
NQLQNSGWNLDSKAVAGSSGKVISGNVSPSKGKMDETVNINAGNNIEITRNGKNIDIATSMTPQ
FSSVSLGAGADAPTLSVDGDALNVGSKKDNKPVRITNVAPGVKEGDVTNVAQLKGVAQNLNN
RIDNVDGNARAGIAQAIATAGLVQAYLPGKSMMAIGGGTYRGEAGYAIGYSSISDGGNWIIKGT
ASGNSRGHFGASASVGYQW
>SEQ ID NO: 6 [App]
MKTTDKRTTETHRKAPKTGRIRFSPAYLAICLSFGILPQAWAGHTYFGINYQYYRDFAENKGKF
AVGAKDIEVYNKKGELVGKSMTKAPMIDFSVVSRNGVAALVGDQYIVSVAHNGGYNNVDFGAE
GRNPDQHRFTYKIVKRNNYKAGTKGHPYGGDYHMPRLHKFVTDAEPVEMTSYMDGRKYIDQN
NYPDRVRIGAGRQYWRSDEDEPNNRESSYHIASAYSWLVGGNTFAQNGSGGGTVNLGSEKIK
HSPYGFLPTGGSFGDSGSPMFIYDAQKQKWLINGVLQTGNPYIGKSNGFQLVRKDWFYDEIFA
GDTHSVFYEPRQNGKYSFNDDNNGTGKINAKHEHNSLPNRLKTRTVQLFNVSLSETAREPVYH
AAGGVNSYRPRLNNGENISFIDEGKGELILTSNINQGAGGLYFQGDFTVSPENNETWQGAGVHI
SEDSTVTWKVNGVANDRLSKIGKGTLHVQAKGENQGSISVGDGTVILDQQADDKGKKQAFSEI
GLVSGRGTVQLNADNQFNPDKLYFGFRGGRLDLNGHSLSFHRIQNTDEGAMIVNHNQDKEST
VTITGNKDIATTGNNNSLDSKKEIAYNGWFGEKDTTKTNGRLNLVYQPAAEDRTLLLSGGTNLN
GNITQTNGKLFFSGRPTPHAYNHLNDHWSQKEGIPRGEIVWDNDWINRTFKAENFQIKGGQAV
VSRNVAKVKGDWHLSNHAQAVFGVAPHQSHTICTRSDWTGLTNCVEKTITDDKVIASLTKTDIS
GNVDLADHAHLNLTGLATLNGNLSANGDTRYTVSHNATQNGNLSLVGNAQATFNQATLNGNT
SASGNASFNLSDHAVQNGSLTLSGNAKANVSHSALNGNVSLADKAVFHFESSRFTGQISGGKD
TALHLKDSEWTLPSGTELGNLNLDNATITLNSAYRHDAAGAQTGSATDAPRRRSRRSRRSLLS
VTPPTSVESRFNTLTVNGKLNGQGTFRFMSELFGYRSDKLKLAESSEGTYTLAVNNTGNEPAS
LEQLTVVEGKDNKPLSENLNFTLQNEHVDAGAWRYQLIRKDGEFRLHNPVKEQELSDKLGKAE
AKKQAEKDNAQSLDALIAAGRDAVEKTESVAEPARQAGGENVGIMQAEEEKKRVQADKDTALA
KQREAETRPATTAFPRARRARRDLPQLQPQPQPQPQRDLISRYANSGLSEFSATLNSVFAVQD
ELDRVFAEDRRNAVWTSGIRDTKHYRSQDFRAYRQQTDLRQIGMQKNLGSGRVGILFSHNRT
ENTFDDGIGNSARLAHGAVFGQYGIDRFYIGISAGAGFSSGSLSDGIGGKIRRRVLHYGIQARYR
AGFGGFGIEPHIGATRYFVQKADYRYENVNIATPGLAFNRYRAGIKADYSFKPAQHISITPYLSLS
YTDAASGKVRTRVNTAVLAQDFGKTRSAEWGVNAEIKGFTLSLHAAAAKGPQLEAQHSAGIKL
GYRW
>SEQ ID NO: 7 [NadA fragment]
ATNDDDVKKAATVAIAAAYNNGQEINGFKAGETIYDIDEDGTITKKDATAADVEADDFKGLGLKK
VVTNLTKTVNENKQNVDAKVKAAESEIEKLTTKLADTDAALADTDAALDATTNALNKLGENITTF
AEETKTNIVKIDEKLEAVADTVDKHAEAFNDIADSLDETNTKADEAVKTANEAKQTAEETKQNVD
AKVKAAETAAGKAEAAAGTANTAADKAEAVAAKVTDIKADIATNKDNIAKKANSADVYTREESD
SKFVRIDGLNATTEKLDTRLASAEKSIADHDTRLNGLDKTVSDLRKETRQGLAEQAALSGLFQP
YNVG
>SEQ ID NO: 8 [fHbp v1]
VAADIGAGL ADALTAPLDH KDKGLQSLTL DQSVRKNEKL KLAAQGAEKT YGNGDSLNTG
KLKNDKVSRF DFIRQIEVDG QLITLESGEF QVYKQSHSAL TAFQTEQIQD SEHSGKMVAK
RQFRIGDIAG EHTSFDKLPE GGRATYRGTA FGSDDAGGKL TYTIDFAAKQ GNGKIEHLKS
PELNVDLAAA DIKPDGKRHA VISGSVLYNQ AEKGSYSLGI FGGKAQEVAG SAEVKTVNGI
RHIGLAAKQ
>SEQ ID NO: 9 [fHbp v3]
VAADIGTGLADALTAPLDHKDKGLKSLTLEDSIPQNGTLTLSAQGAEKTFKAGDKDNSLNTGKL
KNDKISRFDFVQKIEVDGQTITLASGEFQIYKQNHSAVVALQIEKINNPDKTDSLINQRSFLVSGL
GGEHTAFNQLPGGKAEYHGKAFSSDDPNGRLHYSIDFTKKQGYGRIEHLKTLEQNVELAAAEL
KADEKSHAVILGDTRYGSEEKGTYHLALFGDRAQEIAGSATVKIGEKVHEIGIAGKQ
>SEQ ID NO: 10 [Pfs25]
KVTVDTVCKR GFLIQMSGHL ECKCENDLVL VNEETCEEKV LKCDEKTVNK PCGDFSKCIK
IDGNPVSYAC KCNLGYDMVN NVCIPNECKQ VTCGNGKCIL DTSNPVKTGV CSCNIGKVPN
VQDQNKCSKD GETKCSLKCL KEQETCKAVD GIYKCDCKDG FIIDQESSIC T
>SEQ ID NO: 11 [RO6C]
AERSTSENRNKRIGGPKLRGNVTSNIKFPSDNKGKIIRGSNDKLNKNSEDVLEQSEKSLVSENV
PSGLDIDDIPKESIFIQEDQEGQTHSELNPETSEHSKDLNNNGSKNESSDIISENNKSNKVQNHF
ESLSDLELLENSSQDNLDKDTISTEPFPNQKHKDLQQDLNDEPLEPFPTQIHKDYKEKNLINEED
SEPFPRQKHKKVDNHNEEKNVFHENGSANGNQGSLKLKSFDEHLKDEKIENEPLVHENLSIPN
DPIEQILNQPEQETNIQEQLYNEKQNVEEKQNSQIPSLDLKEPTNEDILPNHNPLENIKQSESEIN
HVQDHALPKENIIDKLDNQKEHIDQSQHNINVLQENNINNHQLEPQEKPNIESFEPKNIDSEIILPE
NVETEEIIDDVPSPKHSNHETFEEETSESEHEEAVSEKNAHETVEHEETVSQESNPEKADNDG
NVSQNSNNELNENEFVESEKSEHEARSKPKYEKKVIHGCNFSSNVSSKHTFTDSLDISLVDDSA
HISCNVHLSEPKYNHLVGLNCPGDIIPDCFFQVYQPESEELEPSNIVYLDSQINIGDIEYYEDAEG
DDKIKLFGIVGSIPKTTSFTCICKKDKKSAYMTVTIDSARSHHHHHH
>SEQ ID NO: 12 [CSP]
MLFQEYQCYGSSSNTRVLNELNYDNAGTNLYNELEMNYYGKQENWYSLKKNSRSLGENDDG
NNNNGDNGREGKDEDKRDGNNEDNEKLRKPKHKKLKQPGDGNPDPNANPNVDPNANPNVD
PNANPNVDPNANPNANPNANPNANPNANPNANPNANPNANPNANPNANPNANPNANPNANP
NANPNANPNVDPNANPNANPNANPNANPNANPNANPNANPNANPNANPNANPNANPNANPN
ANPNANPNANPNANPNANPNANPNANPNKNNQGNGQGHNMPNDPNRNVDENANANNAVKN
NNNEEPSDKHIEKYLKKIQNSLSTEWSPCSVTCGNGIQVRIKPGSANKPKDELDYENDIEKKICK
MEKCSSVFNVVNSSIGLILEHHHHHH
>SEQ ID NO: 13 [(NANP)3]
NANPNANPNANP
>SEQ ID NO: 14 [CTF1232]
QDQRYISIRNTDTIWLPGNICAYQFRLDNGGNDEGFGPLTITLQLKDKYGQTLVTRKMETEAFG
DSNATRTTDAFLETECVENVATTEIIKATEESNGHRVSLPLSVFDPQDYHPLLITVSGKNVNLEH
HHHHH
>SEQ ID NO: 15 [3526, SsIE]
DTPSVDSGSGTLPEVKPDPTPTPEPTPEPTPDPEPTPDPTPDPEPTPEPEPEPVPTKTGYLTLG
GSQRVTGATCNGESSDGFTFTPGNTVSCVVGSTTIATFNTQSEAARSLRAVDKVSFSLEDAQE
LANSENKKTNAISLVTSSDSCPADAEQLCLTFSSWDRARFEKLYKQIDLATDNFSKLVNEEVEN
NAATDKAPSTHTSTVVPVTTEGTKPDLNASFVSANAEQFYQYQPTEIILSEGQLVDSLGNGVAG
VDYYTNSGRGVTDENGKFSFSWGETISFGIDTFELGSVRGNKSTIALTELGDEVRGANIDQLIH
RYSTTGQNNTRVVPDDVRKVFAEYPNVINEIINLSLSNGATLDEGDQNVVLPNEFIEQFKTGQA
KEIDTAICAKTDGCNEARWFSLTTRNVNDGQIQGVINKLWGVDTNYQSVSKFHVFHDSTNFYG
STGNARGQAVVNISNSAFPILMARNDKNYWLAFGEKRAWDKNELAYITEAPSIVQPENVT
RDTATFNLPFISLGQVGEGKLMVIGNPHYNSILRCPNGYSWGGGVNSKGECTLSGDSDDM
KHFMQNVLRYLSNDIWQPNTKSIMTVGTNLENVYFKKAGQVLGNSAPFAFHEDFTGITVK
QLTSYGDLNPEEIPLLILNGFEYVTQWSGDPYAVPLRADTSKPKLTQQDVTDLIAYLNKG
GSVLIMENVMSNLKEESASSFVRLLDAAGLSMALNKSWNNDPQGYPDRVRQRRATGIWV
YERYPAADGAQPPYTIDPNTGEVTWKYQQDNKPDDKPKLEVASWQEEVEGKQVTRYAFID
EAEYTTEESLEAAKAKIFEKFPGLQECKDSTYHYEINCLERRPGTDVPVTGGMYVPRYTQ
LNLDADTAKAMVQAADLGTNIQRLYQHELYFRTKGSKGERLNSVDLERLYQNMSVWLWND
TKYRYEEGKEDELGFKTFTEFLNCYANDAYAGGTKCSADLKKSLVDNNMIYGDGSSKAGM
MNPSYPLNYMEKPLTRLMLGRSWWDLNIKVDVEKYPGSVSAKGESVTENISLYSNPTKWF
AGNMQSTGLWAPAQQDVTIKSSASVPVTVTVALADDLTGREKHEVALNRPPRVTKTYTLE
ANGEVTFKVPYGGLIYIKGDSKDDVSANFTFTGVVKAPFYKDGEWKNDLDSPAPLGELES
ASFVYTTPKKNLEASNFTGGVAEFAKDLDTFASSMNDFYGRNDEDGKHRMFTYKNLTGHK
HRFTNDVQISIGDAHSGYPVMNSSFSTNSTTLPTTPLNDWLIWHEVGHNAAETPLNVPGA
TEVANNVLALYMQDRYLGKMNRVADDITVAPEYLDESNGQAWARGGAGDRLLMYAQLKEW
AEENFDIKQWYPDGELPKFYSDRKGMKGWNLFQLMHRKARGDDVGNSTFGGKNYCAESNG
NAADTLMLCASWVAQADLSEFFKKWNPGASAYQLPGATEMSFQGGVSSSAYSTLASLKLP
KPEKGPETINKVTEHKMSAE
>SEQ ID NO: 16 [405, FdeC]
VADGQQAYTLTLTAVDSEGNPVTGEASRLRLVPQDTNGVTVGAISEIKPGVYSATVSSTR
AGNVVVRAFSEQYQLGTLQQTLKFVAGPLDAAHSSITLNPDKPVVGGTVTAIWTAKDAND
NPVTGLNPDAPSLSGAAAAGSTASGWTDNGDGTWTAQISLGTTAGELDVMPKLNGQDAAA
NAAKVTVVADALSSNQSKVSVAEDHVKAGESTTVTLVAKDAHGNAISGLSLSASLTGTAS
EGATVSSWTEKGDGSYVATLTTGGKTGELRVMPLFNGQPAATEAAQLTVIAGEMSSANST
LVADNKTPTVKTTTELTFTMKDAYGNPVTGLKPDAPVFSGAASTGSERPSAGNWTEKGNG
VYVSTLTLGSAAGQLSVMPRVNGQNAVAQPLVLNVAGDASKAEIRDMTVKVNNQ

1. An immunogenic conjugate comprising a native outer membrane vesicle (nOMV), having at least a surface saccharide moiety connected to an antigen, and at least a surface protein residue connected to a different antigen through a bivalent linker.

2. The derivative according to claim 1, wherein said bivalent Linker has the general formula (I):
X-L-X′ (I)
wherein:
X and X′ are different to each other or the same, and are a functional group able to selectively react with the nOMV protein residue on one hand and with the antigen on the other hand,
-L- is a bivalent linear or branched C₁-C₁₅ alkyl or alkenyl group optionally substituted, and optionally interrupted by one or more heteroatom selected from: oxygen (—O—), sulfur (—S—), nitrogen (—NH— or optionally substituted —N— group) and the like.

3. The conjugate according to claim 2, wherein said bivalent Linker is a homobifunctional Linker having X═X′.

4. The conjugate according to claim 2 or 3, wherein said bivalent Linker is selected from at least one of: disuccinimidyl glutarate (DSG), disuccinimidyl suberate (DSS), succinimidyl 3-(2-pyridyldithio)propionate (SPDP), Succinimidyl 6-(3-[2-pyridyldithio]-propionamido)hexanoate (LC-SPDP), sulfosuccinimidyl 6-(3′-(2-pyridyldithio)propionamido)hexanoate (sulfo-LC-SPDP), 4-succinimidyloxycarbonyl-α-methyl-α-(2-pyridyldithio)toluene (SMPT), sulfosuccinimidyl-6-[α-methyl-α-(2-pyridyldithio)tolueamideo]hexanoate (sulfo-LC-SMPT), succinimidyl 4-(N-maleimidomethyl)cyclohexane-1-carboxylate (SMCC), sulfosuccinimidyl 4-(N-maleimidomethyl)cyclohexane-1-carboxylate (suflo-SMCC), m-maleimidobenzoyl-N-hydroxysuccinimide ester (MBS), m-maleimidobenzoyl-N-hydroxysulfosuccinimide ester (sulfo-MBS), N-succinimidyl (4-iodoacetyl)aminobenzoate (SIAB), sulfosuccinimidyl (4-iodoacetyl)aminobenzoate (sulfo-SIAB), succinimidyl 4-(N-maleimidophenyl)butyrate (SMPB), sulfosuccinimidyl 4-(N-maleimidophenyl)butyrate(sulfo-SMPB), N-γ-maleimidobutyryl-oxysuccinimide ester (GMBS), N-γ-maleimidobutyryl-oxysulfosuccinimide ester (sulfo-GMBS), succinimidyl-6-((((4-(iodoacetyl)amino)methyl)cyclohexane-1-carbonyl)amino)hexanoate (SIACX), succinimidyl 6[6-(((iodoacetyl)amino)hexanoyl)amino]hexanoate (SIAXX), succinimidyl-4-(((iodoacetyl)amino)methyl)cyclohexane-1-carboxylate (SIAC), succinimidyl 6-[(iodoacetyl)amino]hexanoate (SIAX) and β-nitrophenyl iodoacetate (NPIA), N-hydroxysuccinimide, N oxysuccinimide and adipic acid N-hydroxysuccinimide diester (SIDEA) and Bis(sulfosuccinimidyl) suberate (BS3, CAS No. 82436-77-9), acryloyl halides (e.g. chloride), ethylene glycol bis[succinimidylsuccinate], bis(sulfosuccinimidyl)tri(ethylene glycol) (BS(PEG)3), bis(sulfosuccinimidyl)tetra(ethylene glycol) (BS(PEG)4), bis(sulfosuccinimidyl)penta(ethylene glycol) (BS(PEG)5) and bis(sulfosuccinimidyl)exa(ethylene glycol) (BS(PEG)6), adipic acid dihydrazide (ADH), β-propionamido, nitrophenyl-ethylamine, haloacyl halides, and 6-aminocaproic acid.

5. The conjugate according to claim 4, wherein said bivalent Linker is BS3 or SIDEA.

6. The conjugate according to claims 1-5, wherein said nOMV is obtained by a detergent free process, being released into the fermentation broth and purified using a centrifugation and subsequent filtration; or being released into the fermentation broth and purified using two consecutive Tangential Flow Filtration (TFF) steps.

7. The conjugate according to any one of the preceding claims, wherein said nOMV is obtained from a bacteria selected from: Neisseria, Shigella, Salmonella enterica serovars, Haemophilus influenzae, Vibrio cholerae, Bordetella pertussis, Mycobacterium smegmatis, Mycobacterium bovis BCG, Escherichia coli, Bacteroides, Pseudomonas aeruginosa, Helicobacter pylori, Brucella melitensis Campylobacter jejuni, Actinobacillus actinomycetemcomitans, Xenorhabdus nematophilus, Moraxella catarrhalis, or Borrelia burgdorferi.

8. The conjugate according to any one of the preceding claims, wherein the antigens are selected from an immunogenic polypeptide or a capsular polysaccharide.

9. The conjugate according to any one of the preceding claims, wherein said nOMV and the antigens connected through a surface saccharide moiety and through a bivalent linker respectively are derived from different bacterial strain.

10. The conjugate according to any one of the preceding claims, wherein said nOMV and the antigen connected through a surface saccharide moiety are selected as follows:

| nOMV | Antigen |
|---|---|
| Salmonella Typhimurium | Neisseria meningitidis fHbp |
| Salmonella Typhimurium | Plasmodium falciparum CSP |
| Salmonella Typhimurium | Plasmodium falciparum Pfs25 |
| Salmonella Typhimurium | Plasmodium falciparum RO6C |
| Salmonella Typhimurium | Plasmodium falciparum RO10C |
| Salmonella Typhimurium | Escherichia coli CTF1232 |
| Salmonella Typhimurium | S. Typhi Vi saccharide |
| Neisseria meningitidis | Neisseria meningitidis fHbp |
| Neisseria meningitidis | Neisseria meningitidis NHBA |
| Neisseria meningitidis | Poly-rhamnose oligosaccharide |
| Shigella | Escherichia coli CTF1232 |
| Shigella | Escherichia coli FdeC |
| Shigella | Escherichia coli SsIE |
| Salmonella Typhimurium | Synthetic or native GAS PS |
| Neisseria meningitidis | Synthetic or native GAS PS |
| Salmonella Typhimurium | Synthetic or native GBS PS |
| Neisseria meningitidis, | Neisseria meningitidis ser A saccharide |
| preferably B | |
| Neisseria meningitidis, | Neisseria meningitidis ser C saccharide |
| preferably B | |
| Salmonella Typhimurium | Neisseria meningitidis ser A saccharide |
| Salmonella Typhimurium | Neisseria meningitidis ser C saccharide |
| B. pertussis | Haemophilus influenzae type b |
| Neisseria meningitidis, | Haemophilus influenzae type b |
| preferably B | |
| B. pertussis | Haemophilus influenzae type a |
| Neisseria meningitidis, | Haemophilus influenzae type a |
| preferably B | |
| Salmonella Typhimurium | Streptococcus pneumoniae saccharide |
| Neisseria meningitidis | Streptococcus pneumoniae saccharide |

11. The conjugate according to any one of the preceding claims, wherein said nOMV, bivalent Linker connected to the nOMV surface protein and the antigen are selected as follows:

| nOMV | Linker | Antigen |
|---|---|---|
| Salmonella Typhimurium | BS3 | Plasmodium falciparum Pfs25 |
| Salmonella Typhimurium | BS(PEG)5 | Plasmodium falciparum Pfs25 |
| Salmonella Typhimurium | BS3 | fHbp (Neisseria meningitidis) |
| Salmonella Typhimurium | BS(PEG)5 | fHbp (Neisseria meningitidis) |
| Salmonella Typhimurium | BS3 | Plasmodium falciparum RO6C |
| Salmonella Typhimurium | BS(PEG)5 | Plasmodium falciparum RO6C |
| Salmonella Typhimurium | BS3 | Plasmodium falciparum CSP |
| Salmonella Typhimurium | BS(PEG)5 | Plasmodium falciparum CSP |
| Meningococcal B | BS3 | fHbp (Neisseria meningitidis) |
| Meningococcal B | BS(PEG)5 | fHbp (Neisseria meningitidis) |
| Meningococcal B | BS3 | NHBA (Neisseria meningitidis) |
| Meningococcal B | BS(PEG)5 | NHBA (Neisseria meningitidis) |
| Salmonella Typhimurium | BS3 | Synthetic or native GAS PS |
| Salmonella Typhimurium | BS(PEG)5 | Synthetic or native GAS PS |
| Salmonella Typhimurium | BS3 | Synthetic or native GBS PS |
| Meningococcal B | BS3 | Synthetic or native GAS PS |
| Meningococcal B | BS3 | Hib PS |
| B. pertussis | BS3 | Hib PS |
| Meningococcal B | BS3 | Hia PS |
| B. pertussis | BS3 | Hia PS |
| Salmonella Typhimurium | BS3 | Vi PS |
| Shigella | BS3 | E. coli FdeC |
| Shigella | BS3 | E. coli SsIE |
| Shigella | BS3 | E. coli CTF1232 |
| Meningococcal B | BS3 or SIDEA | Neisseria meningitidis ser C |
| | | saccharide |
| Meningococcal B | BS3 or SIDEA | Neisseria meningitidis ser A |
| | | saccharide |
| Salmonella Typhimurium | BS3 or SIDEA | Neisseria meningitidis ser C |
| | | saccharide |
| Salmonella Typhimurium | BS3 or SIDEA | Neisseria meningitidis ser A |
| | | saccharide |
| Salmonella Typhimurium | BS3 or SIDEA | Streptococcus pneumoniae |
| | | saccharide |
| Neisseria meningitidis | BS3 or SIDEA | Streptococcus pneumoniae |
| | | saccharide |
| nOMV | Antigen 1 (via PS) | Antigen2/linker |

12. The conjugate according to any one of the preceding claims, functionalized as follow:

| | | |
|---|---|---|
| Salmonella Typhimurium | Pfs25 | (NANP)3 |
| Meningococcal B | MenA | MenC/SIDEA or BS3 |
| Meningococcal B | MenC | MenA/SIDEA or BS3 |
| Salmonella Typhimurium | MenA | MenC/SIDEA or BS3 |
| Salmonella Typhimurium | MenC | MenA/SIDEA or BS3 |
| Salmonella Typhimurium | Native or synthetic GAS PS | Neisseria meningitidis fHbp/BS3 |
| Salmonella Typhimurium | Neisseria meningitidis fHbp | Native or synthetic GAS PS/BS3 |
| Meningococcal B | Hia PS | Hib PS/SIDEA or BS3 |
| B. Pertussis | Hia PS | Hib PS/SIDEA or BS3 |
| Salmonella Typhimurium | Hia PS | Hib PS/SIDEA or BS3 |
| Meningococcal B | Hib PS | Hia PS/SIDEA or BS3 |
| B. Pertussis | Hib PS | Hia PS/SIDEA or BS3 |
| Salmonella Typhimurium | Hib PS | Hia PS/SIDEA or BS3 |
| Shigella | SsIE | FdeC/BS3 |
| Shigella | FdeC | SsIE/BS3 |

13. The conjugate according to claim 13 selected from:

| | | |
|---|---|---|
| Meningococcal B | MenA | MenC/SIDEA or BS3 |
| Meningococcal B | MenC | MenA/SIDEA or BS3 |

14. The conjugate according to claim 13, selected from:

| | | |
|---|---|---|
| Shigella | SsIE | FdeC/BS3 |
| Shigella | FdeC | SsIE/BS3 |

15. The conjugate according to any one of the preceding claims, wherein said nOMV is produced from wild type bacteria or from genetically-modified bacterial strains that are mutated to enhance vesicle production.

16. A process for preparing the conjugate according to any one of claims 1-15, comprising the steps of:
i) activating at least a nOMV saccharide moiety, generally bond to the nOMV surface, and ii) connecting the thus obtained activated saccharide to at least one antigen to obtain an antigen-nOMV intermediate;
iii) reacting at least a nMOV surface protein residue of said antigen-nOMV intermediate with the first terminal portion of a bivalent Linker, to obtain an antigen-nOMV-Linker intermediate, and
iv) connecting the thus obtained antigen-nOMV-Linker intermediate to at least a different antigen via the second terminal portion of the bivalent Linker, thus obtaining the antigen-nOMV-Linker-antigen derivative of the invention;
or
i) reacting at least a nMOV surface protein residue with the first terminal portion of a bivalent Linker to obtain a nOMV-Linker intermediate, and
ii) connecting said nOMV-Linker intermediate to at least one antigen via the second terminal portion of the bivalent Linker, thus obtaining a nOMV-Linker-antigen intermediate,
iii) activating at least a nOMV saccharide moiety of the thus obtained nOMV-Linker-antigen intermediate, and
v) connecting the thus obtained activated saccharide to at least a different antigen, to obtain the antigen-nOMV-Linker-antigen derivative of the invention.

17. An immunogenic composition comprising a conjugate according to any one of claims 1-15 and at least one pharmaceutically acceptable carrier or excipient.

18. A conjugate or composition according to any one of claims 1-15 or 17, for use as medicament.

19. A conjugate or composition for use according to claim 18 to induce an immune response in a vertebrate.


SEQUENCE LISTING (16 sequences)

SEQ ID NO: 1 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFRVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 2 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 3 - protein, 362 aa, Neisseria meningitidis
MKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 4 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 5 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 6 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 7 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 8 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 9 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 10 - protein, 171 aa, Synthetic construct
  source 1..171: note = P.falciparum
KVTVDTVCKRGFLIQMSGHLECKCENDLVLVNEETCEEKVLKCDEKTVNKPCGDFSKCIKIDGNPVSYACKCNLGYDMVNNVCIPNECKQVTCGNGKCILDTSNPVKTGVCSCNIGKVPNVQDQNKCSKDGETKCSLKCLKEQETCKAVDGIYKCDCKDGFIIDQESSICT

SEQ ID NO: 11 - protein, 630 aa, Synthetic construct
  REGION 1..630: note = pfs48/45-GURLP fusion protein
AERSTSENRNKRIGGPKLRGNVTSNIKFPSDNKGKIIRGSNDKLNKNSEDVLEQSEKSLVSENVPSGLDIDDIPKESIFIQEDQEGQTHSELNPETSEHSKDLNNNGSKNESSDIISENNKSNKVQNHFESLSDLELLENSSQDNLDKDTISTEPFPNQKHKDLQQDLNDEPLEPFPTQIHKDYKEKNLINEEDSEPFPRQKHKKVDNHNEEKNVFHENGSANGNQGSLKLKSFDEHLKDEKIENEPLVHENLSIPNDPIEQILNQPEQETNIQEQLYNEKQNVEEKQNSQIPSLDLKEPTNEDILPNHNPLENIKQSESEINHVQDHALPKENIIDKLDNQKEHIDQSQHNINVLQENNINNHQLEPQEKPNIESFEPKNIDSEIILPENVETEEIIDDVPSPKHSNHETFEEETSESEHEEAVSEKNAHETVEHEETVSQESNPEKADNDGNVSQNSNNELNENEFVESEKSEHEARSKPKYEKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSARSHHHHHH

SEQ ID NO: 12 - protein, 394 aa, Synthetic construct
  source 1..394: note = circumsporozoite
MLFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEKYLKKIQNSLSTEWSPCSVTCGNGIQVRIKPGSANKPKDELDYENDIEKKICKMEKCSSVFNVVNSSIGLILEHHHHHH

SEQ ID NO: 13 - protein, 12 aa, Synthetic construct
  source 1..12: note = circumsporozoite
NANPNANPNANP

SEQ ID NO: 14 - protein, 134 aa, Synthetic construct
  source 1..134: note = E. coli
QDQRYISIRNTDTIWLPGNICAYQFRLDNGGNDEGFGPLTITLQLKDKYGQTLVTRKMETEAFGDSNATRTTDAFLETECVENVATTEIIKATEESNGHRVSLPLSVFDPQDYHPLLITVSGKNVNLEHHHHHH

SEQ ID NO: 15 - protein, 1487 aa, Synthetic construct
  source 1..1487: note = E.coli
DTPSVDSGSGTLPEVKPDPTPTPEPTPEPTPDPEPTPDPTPDPEPTPEPEPEPVPTKTGYLTLGGSQRVTGATCNGESSDGFTFTPGNTVSCVVGSTTIATFNTQSEAARSLRAVDKVSFSLEDAQELANSENKKTNAISLVTSSDSCPADAEQLCLTFSSVVDRARFEKLYKQIDLATDNFSKLVNEEVENNAATDKAPSTHTSTVVPVTTEGTKPDLNASFVSANAEQFYQYQPTEIILSEGQLVDSLGNGVAGVDYYTNSGRGVTDENGKFSFSWGETISFGIDTFELGSVRGNKSTIALTELGDEVRGANIDQLIHRYSTTGQNNTRVVPDDVRKVFAEYPNVINEIINLSLSNGATLDEGDQNVVLPNEFIEQFKTGQAKEIDTAICAKTDGCNEARWFSLTTRNVNDGQIQGVINKLWGVDTNYQSVSKFHVFHDSTNFYGSTGNARGQAVVNISNSAFPILMARNDKNYWLAFGEKRAWDKNELAYITEAPSIVQPENVTRDTATFNLPFISLGQVGEGKLMVIGNPHYNSILRCPNGYSWGGGVNSKGECTLSGDSDDMKHFMQNVLRYLSNDIWQPNTKSIMTVGTNLENVYFKKAGQVLGNSAPFAFHEDFTGITVKQLTSYGDLNPEEIPLLILNGFEYVTQWSGDPYAVPLRADTSKPKLTQQDVTDLIAYLNKGGSVLIMENVMSNLKEESASSFVRLLDAAGLSMALNKSVVNNDPQGYPDRVRQRRATGIWVYERYPAADGAQPPYTIDPNTGEVTWKYQQDNKPDDKPKLEVASWQEEVEGKQVTRYAFIDEAEYTTEESLEAAKAKIFEKFPGLQECKDSTYHYEINCLERRPGTDVPVTGGMYVPRYTQLNLDADTAKAMVQAADLGTNIQRLYQHELYFRTKGSKGERLNSVDLERLYQNMSVWLWNDTKYRYEEGKEDELGFKTFTEFLNCYANDAYAGGTKCSADLKKSLVDNNMIYGDGSSKAGMMNPSYPLNYMEKPLTRLMLGRSWWDLNIKVDVEKYPGSVSAKGESVTENISLYSNPTKWFAGNMQSTGLWAPAQQDVTIKSSASVPVTVTVALADDLTGREKHEVALNRPPRVTKTYTLEANGEVTFKVPYGGLIYIKGDSKDDVSANFTFTGVVKAPFYKDGEWKNDLDSPAPLGELESASFVYTTPKKNLEASNFTGGVAEFAKDLDTFASSMNDFYGRNDEDGKHRMFTYKNLTGHKHRFTNDVQISIGDAHSGYPVMNSSFSTNSTTLPTTPLNDWLIWHEVGHNAAETPLNVPGATEVANNVLALYMQDRYLGKMNRVADDITVAPEYLDESNGQAWARGGAGDRLLMYAQLKEWAEENFDIKQWYPDGELPKFYSDRKGMKGWNLFQLMHRKARGDDVGNSTFGGKNYCAESNGNAADTLMLCASWVAQADLSEFFKKWNPGASAYQLPGATEMSFQGGVSSSAYSTLASLKLPKPEKGPETINKVTEHKMSAE

SEQ ID NO: 16 - protein, 414 aa, Synthetic construct
  source 1..414: note = E.coli
VADGQQAYTLTLTAVDSEGNPVTGEASRLRLVPQDTNGVTVGAISEIKPGVYSATVSSTRAGNVVVRAFSEQYQLGTLQQTLKFVAGPLDAAHSSITLNPDKPVVGGTVTAIWTAKDANDNPVTGLNPDAPSLSGAAAAGSTASGWTDNGDGTWTAQISLGTTAGELDVMPKLNGQDAAANAAKVTVVADALSSNQSKVSVAEDHVKAGESTTVTLVAKDAHGNAISGLSLSASLTGTASEGATVSSWTEKGDGSYVATLTTGGKTGELRVMPLFNGQPAATEAAQLTVIAGEMSSANSTLVADNKTPTVKTTTELTFTMKDAYGNPVTGLKPDAPVFSGAASTGSERPSAGNWTEKGNGVYVSTLTLGSAAGQLSVMPRVNGQNAVAQPLVLNVAGDASKAEIRDMTVKVNNQ